SEQUENCE LISTING (63 sequences)

SEQ ID NO: 1 - DNA, 1347 bp, Coronavirus
atgtcttttactcctggtaagcaatccagtagtagagcgtcctctggaaatcgttctggtaatggcatcctcaagtgggccgatcagtccgaccagtttagaaatgttcaaaccaggggtagaagagctcaacccaagcaaactgctacctctcagcaaccatcaggagggaatgttgtaccctactattcttggttctctggaattactcagtttcaaaagggaaaggagtttgagtttgtagaaggacaaggtgtgcctattgcaccaggagtcccagctactgaagctaaggggtactggtacagacacaacagacgttcttttaaaacagccgatggcaaccagcgtcaactgctgccacgatggtatttttactatctgggaacaggaccgcatgctaaagaccagtacggcaccgatattgacggagtctactgggtcgctagcaaccaggctgatgtcaataccccggctgacattgtcgatcgggacccaagtagcgatgaggctattccgactaggtttccgcctggcacggtactccctcagggttactatattgaaggctcaggaaggtctgctcctaattccagatctacttcgcgcacatccagcagagcctctagtgcaggatcgcgtagtagagccaattctggcaatagaacccctacctctggtgtaacacctgacatggctgatcaaattgctagtcttgttctggcaaaacttggcaaggatgccactaaacctcagcaagtaactaagcatactgccaaagaagtcagacagaaaattttgaataagccccgccagaagaggagccccaataaacaatgcactgttcagcagtgttttggtaagagaggccctaatcagaattttggtggtggagaaatgttaaaacttggaactagtgacccacagttccccattcttgcagaactcgcacccacagctggtgcgtttttctttggatcaagattagagttggccaaagtgcagaatttatctgggaatcctgacgagccccagaaggatgtttatgaattgcgctataacggcgcaattaggtttgacagtacactttcaggttttgagaccataatgaaggtgctgaatgagaatttgaatgcctatcaacaacaagatggtatgatgaatatgagtccaaaaccacagcgtcagcgtggtcataagaatggacaaggagaaaatgataatataagtgttgcagtgcccaaaagccgcgtgcagcaaaataagagtagagagttgactgcagaggacatcagccttcttaagaagatggatgagccctatactgaagacacctcagaaatataa

SEQ ID NO: 2 - protein, 448 aa, coronavirus
MSFTPGKQSSSRASSGNRSGNGILKWADQSDQFRNVQTRGRRAQPKQTATSQQPSGGNVVPYYSWFSGITQFQKGKEFEFVEGQGVPIAPGVPATEAKGYWYRHNRRSFKTADGNQRQLLPRWYFYYLGTGPHAKDQYGTDIDGVYWVASNQADVNTPADIVDRDPSSDEAIPTRFPPGTVLPQGYYIEGSGRSAPNSRSTSRTSSRASSAGSRSRANSGNRTPTSGVTPDMADQIASLVLAKLGKDATKPQQVTKHTAKEVRQKILNKPRQKRSPNKQCTVQQCFGKRGPNQNFGGGEMLKLGTSDPQFPILAELAPTAGAFFFGSRLELAKVQNLSGNPDEPQKDVYELRYNGAIRFDSTLSGFETIMKVLNENLNAYQQQDGMMNMSPKPQRQRGHKNGQGENDNISVAVPKSRVQQNKSRELTAEDISLLKKMDEPYTEDTSEI

SEQ ID NO: 3 - DNA, 1326 bp, coronavirus
atgtcttatactcccggtcattatgctggaagtagaagctcctctggaaatcgttcaggaatcctcaagaaaacttcttgggctgaccaatctgagcgaaattaccaaacctttaatagaggcagaaaaacccaacctaaattcactgtgtctactcaaccacaaggaaatactatcccacattattcctggttctccgggatcactcaatttcaaaaaggtagagactttaaattttcagatggtcaaggagttcccattgctttcggagtacccccttctgaagcaaaaggatattggtatagacacagccggcgttcttttaaaacagctgatggtcaacaaaagcagttgttaccgagatggtatttctactatctcggtaccggcccatatgccaatgcatcctatggtgaatccctcgaaggggtcttctgggttgctaatcaccaagctgacacttctactccctccgatgtttcgtcaagggatcctactactcaagaagctatccctactaggtttccgcctggtacgattttgcctcaaggctattatgttgaaggctcaggaaggtctgcttctaatagtcgaccaggttcacgttctcaatcacgtggacccaataatcgttcattaagtagaagtaattctaattttagacattcagattctatagtaaaacctgatatggctgatgagatcgctaatcttgttttagccaagcttggtaaagattctaaacctcagcaagtcactaagcaaaatgccaaggaaatcaggcataaaattttaacaaaacctcgccaaaagcgaactcctaataaacattgtaatgttcaacagtgttttggtaaaagaggaccttctcaaaattttggtaatgctgaaatgttaaagcttggtactaatgatcctcagtttcctattcttgcagaattagctcctacaccaggtgcttttttctttggttctaaattagacttggttaaaagagattccgaggctgactcacctgttaaagatgtttttgaacttcattattctggttctattaggtttgatagtactttaccaggctttgagacaattatgaaagttcttgaagagaatttaaatgcttacgttaattctaatcagaacactgattctgattcgttgagttctaaacctcagcgtaaaagaggtgttaaacaattaccagaacagtttgactctcttaatttaagtgctggtactcagcacatttcaaatgattttactcctgaggatcatagtttacttgctactcttgatgatccttatgtagaagactctgttgcttaa

SEQ ID NO: 4 - protein, 448 aa, coronavirus; identical to SEQ ID NO: 2

SEQ ID NO: 5 - DNA, 1134 bp, coronaviurs
atggctagtgtaaattgggccgatgacagagctgctaggaagaaatttcctcctccttcattttacatgcctcttttggttagttctgataaggcaccatatagggtcattcccaggaatcttgtccctattggtaagggtaataaagatgagcagattggttattggaatgttcaagagcgttggcgtatgcgcagggggcaacgtgttgatttgcctcctaaagttcatttttattacctaggtactggacctcataaggaccttaaattcagacaacgttctgatggtgttgtttgggttgctaaggaaggtgctaaaactgttaataccagtcttggtaatcgcaaacgtaatcagaaacctttggaaccaaagttctctattgctttgcctccagagctctctgttgttgagtttgaggatcgctctaataactcatctcgtgctagcagtcgttcttcaactcgtaacaactcacgagactcttctcgtagcacttcaagacaacagtctcgcactcgttctgattctaaccagtcttcttcagatcttgttgctgctgttactttggctttaaagaacttaggttttgataaccagtcgaagtcacctagttcttctggtacttccactcctaagaaacctaataagcctctttctcaacccagggctgataagccttctcagttgaagaaacctcgttggaagcgtgttcctaccagagaggaaaatgttattcagtgctttggtcctcgtgattttaatcacaatatgggggattcagatcttgttcagaatggtgttgatgccaaaggttttccacagcttgctgaattgattcctaatcaggctgcgttattctttgatagtgaggttagcactgatgaagtgggtgataatgttcagattacctacacctacaaaatgcttgtagctaaggataataagaaccttcctaagttcattgagcagattagtgcttttactaaacccagttctatcaaagaaatgcagtcacaatcatctcatgttgctcagaacacagtacttaatgcttctattccagaatctaaaccattggctgatgatgattcagccattatagaaattgtcaacgaggttttgcattaa

SEQ ID NO: 6 - protein, 377 aa, coronavirus
MASVNWADDRAARKKFPPPSFYMPLLVSSDKAPYRVIPRNLVPIGKGNKDEQIGYWNVQERWRMRRGQRVDLPPKVHFYYLGTGPHKDLKFRQRSDGVVWVAKEGAKTVNTSLGNRKRNQKPLEPKFSIALPPELSVVEFEDRSNNSSRASSRSSTRNNSRDSSRSTSRQQSRTRSDSNQSSSDLVAAVTLALKNLGFDNQSKSPSSSGTSTPKKPNKPLSQPRADKPSQLKKPRWKRVPTREENVIQCFGPRDFNHNMGDSDLVQNGVDAKGFPQLAELIPNQAALFFDSEVSTDEVGDNVQITYTYKMLVAKDNKNLPKFIEQISAFTKPSSIKEMQSQSSHVAQNTVLNASIPESKPLADDDSAIIEIVNEVLH

SEQ ID NO: 7 - DNA, 1170 bp, coronavirus
atggctacagtcaaatgggctgatgcatctgaaccacaacgtggtcgtcagggtagaataccttattctctttatagccctttgcttgttgatagtgaacaaccttggaaggtgatacctcgtaatttggtacccatcaacaagaaagacaaaaataagcttataggctattggaatgttcaaaaacgtttcagaactagaaagggcaaacgggtggatttgtcacccaagctgcatttttattatcttggcacaggaccccataaagatgcaaaatttagagagcgtgttgaaggtgtcgtctgggttgctgttgatggtgctaaaactgaacctacaggttacggtgttaggcgcaagaattcagaaccagagataccacacttcaatcaaaagctcccaaatggtgttactgttgttgaagaacctgactcccgtgctccttcccggtctcagtcgaggtcgcagagtcgcggtcgtggtgaatccaaacctcaatctcggaatccttcaagtgacagaaaccataacagtcaggatgacatcatgaaggcagttgctgcggctcttaaatctttaggttttgacaagcctcaggaaaaagataaaaagtcagcgaaaacgggtactcctaagccttctcgtaatcagagtcctgcttcttctcaaacttctgccaagagtcttgctcgttctcagagttctgaaacaaaagaacaaaagcatgaaatgcaaaagccacggtggaaaagacagcctaatgatgatgtgacatctaatgtcacacaatgttttggccccagagaccttgaccacaactttggaagtgcaggtgttgtggccaatggtgttaaagctaaaggctatccacaatttgctgagcttgtgccgtcaacagctgctatgctgtttgatagtcacattgtttccaaagagtcaggcaacactgtggtcttgactttcactactagagtgactgtgcccaaagaccatccacacttgggtaagtttcttgaggagttaaatgcattcactagagaaatgcaacaacatcctcttcttaaccctagtgcactagaattcaacccatctcaaacttcacctgcaactgctgaaccagtgcgtgatgaagtttctattgaaactgacataattgatgaagtaaactaa

SEQ ID NO: 8 - protein, 389 aa, coronavirus
MATVKWADASEPQRGRQGRIPYSLYSPLLVDSEQPWKVIPRNLVPINKKDKNKLIGYWNVQKRFRTRKGKRVDLSPKLHFYYLGTGPHKDAKFRERVEGVVWVAVDGAKTEPTGYGVRRKNSEPEIPHFNQKLPNGVTVVEEPDSRAPSRSQSRSQSRGRGESKPQSRNPSSDRNHNSQDDIMKAVAAALKSLGFDKPQEKDKKSAKTGTPKPSRNQSPASSQTSAKSLARSQSSETKEQKHEMQKPRWKRQPNDDVTSNVTQCFGPRDLDHNFGSAGVVANGVKAKGYPQFAELVPSTAAMLFDSHIVSKESGNTVVLTFTTRVTVSKDHPHLGKFLEELNAFTREMQQHPLLNPSALEFNPSQTSPATAEPVRDEVSIETDIIDEVN

SEQ ID NO: 9 - DNA, 1170 bp, coronavirus; identical to SEQ ID NO: 7

SEQ ID NO: 10 - protein, 389 aa, coronavirus
MATVKWADASEPQRGRQGRIPYSLYSPLLVDSEQPWKVIPRNLVPINKKDKNKLIGYWNVQKRFRTRKGKRVDLSPKLHFYYLGTGPHKDAKFRERVEGVVWVAVDGAKTEPTGYGVRRKNSEPEIPHFNQKLPNGVTVVEEPDSRAPSRSQSRSQSRGRGESKPQSRNPSSDRNHNSQDDIMKAVAAALKSLGFDKPQEKDKKSAKTGTPKPSRNQSPASSQTSAKSLARSQSSETKEQKHEMQKPRWKRQPNDDVTSNVTQCFGPRDLDHNFGSAGVVANGVKAKGYPQFAELVPSTAAMLFDSHIVSKESGNTVVLTFTTRVTVPKDHPHLGKFLEELNAFTREMQQHPLLNPSALEFNPSQTSPATAEPVRDEVSIETDIIDEVN

SEQ ID NO: 11 - DNA, 1260 bp, coronavirus
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 12 - protein, 419 aa, coronavirus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 13 - DNA, 4062 bp, coronavirus
atgtttttgatacttttaatttccttaccaacggcttttgctgttataggagatttaaagtgtacttcagataatattaatgataaagacaccggtcctcctcctataagtactgatactgttgatgttactaatggtttgggtacttattatgttttagatcgtgtgtatttaaatactacgttgtttcttaatggttattaccctacttcaggttccacatatcgtaatatggcactgaagggaagtgtactattgagcagactatggtttaaaccaccatttctttctgattttattaatggtatttttgctaaggtcaaaaataccaaggttattaaagatcgtgtaatgtatagtgagttccctgctataactataggtagtacttttgtaaatacatcctatagtgtggtagtacaaccacgtacaatcaattcaacacaggatggtgataataaattacaaggtcttttagaggtctctgtttgccagtataatatgtgcgagtacccacaaacgatttgtcatcctaacctgggtaatcatcgcaaagaactatggcatttggatacaggtgttgtttcctgtttatataagcgtaatttcacatatgatgtgaatgctgattatttgtattttcatttttatcaagaaggtggtactttttatgcatattttacagacactggtgttgttactaagtttttgtttaatgtttatttaggcatggcgctttcacactattatgtcatgcctctgacttgtaatagtaagcttactttagaatattgggttacacctctcacttctagacaatatttactcgctttcaatcaagatggtattatttttaatgctgttgattgtatgagtgattttatgagtgagattaagtgtaaaacacaatctatagcaccacctactggtgtttatgaattaaacggttacactgttcagccaatcgcagatgtttaccgacgtaaacctaatcttcccaattgcaatatagaagcttggcttaatgataagtcggtgccctctccattaaattgggaacgtaagacattttcaaattgtaattttaatatgagcagcctgatgtcttttattcaggcagactcatttacttgtaataatattgatgctgctaagatatatggtatgtgtttttccagcataactatagataagtttgctatacccaatggcaggaaggttgacctacaattgggtaatttgggctatttgcagtcatttaactatagaattgatactactgcaacaagttgtcagttgtattataatttacctgctgctaatgtttctgttagcaggtttaatccttctacttggaataagagatttggttttatagaagattctgtttttaagcctcgacctgcaggtgttcttactaatcatgatgtagtttatgcacaacactgtttcaaagctcctaaaaatttctgtccgtgtaaattgaatggttcgtgtgtaggtagtggtcctggtaaaaataatggtataggcacttgtcctgcaggtactaattatttaacttgtgataatttgtgcactcctgatcctattacatttacaggtacttataagtgcccccaaactaaatctttagttggcataggtgagcactgttcgggtcttgctgttaaaagtgattattgtggaggcaattcttgtacttgccgaccacaagcatttttgggttggtctgcagactcttgtttacaaggagacaagtgtaatatttttgctaattttattttgcatgatgttaatagtggtcttacttgttctactgatttacaaaaagctaacacagacataattcttggtgtttgtgttaattatgacctctatggtattttaggccaaggcatttttgttgaggttaatgcgacttattataatagttggcagaaccttttatatgattctaatggtaatctctacggttttagagactacataacaaacagaacttttatgattcgtagttgctatagcggtcgtgtttctgcggcctttcacgctaactcttccgaaccagcattgctatttcggaatattaaatgcaactacgtttttaataatagtcttacacgacagctgcaacccattaactattttgatagttatcttggttgtgttgtcaatgcttataatagtactgctatttctgttcaaacatgtgatctcacagtaggtagtggttactgtgtggattactctaaaaacagacgaagtcgtggagcgattaccactggttatcggtttactaattttgagccatttactgttaattcagtaaacgatagtttagaacctgtaggtggtttgtatgaaattcaaataccttcagagtttactataggtaatatggtggagtttattcaaacaagctctcctaaagttactattgattgtgctgcatttgtctgtggtgattatgcagcatgtaaatcacagttggttgaatatggtagtttctgtgataacattaatgccatactcacagaagtaaatgaactacttgacactacacagttgcaagtagctaatagtttaatgaatggtgttactcttagcactaagcttaaagatggcgttaatttcaatgtagacgacatcaatttttcccctgtattaggttgtctaggcagcgaatgtagtaaagcttccagtagatctgctatagaggatttactttttgataaagtaaagttatctgatgtcggttttgttgaggcttataataattgtacaggaggtgccgaaattagggacctcatttgtgtgcaaagttataaaggcatcaaagtgttgcctccactgctctcagaaaatcagatcagtggatacactttggctgccacctctgctagtctatttcctccttggacagcagcagcaggtgtaccattttatttaaatgttcagtatcgcattaatgggcttggtgtcaccatggatgtgctaagtcaaaatcaaaagcttattgctaatgcatttaacaatgccctttatgctattcaggaagggttcgatgcaactaattctgctttagttaaaattcaagctgttgttaatgcaaatgctgaagctcttaataacttattgcaacaactctctaatagatttggtgctataagtgcttctttacaagaaattctatctagacttgatgctcttgaagcggaagctcagatagatagacttattaatggtcgtcttaccgctcttaatgcttatgtttctcaacagcttagtgattctacactggtaaaatttagtgcagcacaagctatggagaaggttaatgaatgtgtcaaaagccaatcatctaggataaatttctgtggtaatggtaatcatattatatcattagtgcagaatgctccatatggtttgtattttatccactttagttatgtccctactaagtatgtcacagcgagggttagtcctggtctgtgcattgctggtgatagaggtatagctcctaagagtggttattttgttaatgtaaataatacttggatgtacactggtagtggttactactaccctgaacctataactgaaaataatgttgttgttatgagtacctgcgctgttaattatactaaagcgccgtatgtaatgctgaacacttcaatacccaaccttcctgattttaaggaagagttggatcaatggtttaaaaatcaaacatcagtggcaccagatttgtcacttgattatataaatgttacattcttggacctacaagttgaaatgaataggttacaggaggcaataaaagtcttaaatcagagctacatcaatctcaaggacattggtacatatgaatattatgtaaaatggccttggtatgtatggcttttaatctgccttgctggtgtagctatgcttgttttactattcttcatatgctgttgtacaggatgtgggactagttgttttaagaaatgtggtggttgttgtgatgattatactggataccaggagttagtaatcaaaacttcacatgacgactaa

SEQ ID NO: 14 - protein, 1353 aa, coronavirus
MFLILLISLPTAFAVIGDLKCTSDNINDKDTGPPPISTDTVDVTNGLGTYYVLDRVYLNTTLFLNGYYPTSGSTYRNMALKGSVLLSRLWFKPPFLSDFINGIFAKVKNTKVIKDRVMYSEFPAITIGSTFVNTSYSVVVQPRTINSTQDGDNKLQGLLEVSVCQYNMCEYPQTICHPNLGNHRKELWHLDTGVVSCLYKRNFTYDVNADYLYFHFYQEGGTFYAYFTDTGVVTKFLFNVYLGMALSHYYVMPLTCNSKLTLEYWVTPLTSRQYLLAFNQDGIIFNAVDCMSDFMSEIKCKTQSIAPPTGVYELNGYTVQPIADVYRRKPNLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPRPAGVLTNHDVVYAQHCFKAPKNFCPCKLNGSCVGSGPGKNNGIGTCPAGTNYLTCDNLCTPDPITFTGTYKCPQTKSLVGIGEHCSGLAVKSDYCGGNSCTCRPQAFLGWSADSCLQGDKCNIFANFILHDVNSGLTCSTDLQKANTDIILGVCVNYDLYGILGQGIFVEVNATYYNSWQNLLYDSNGNLYGFRDYITNRTFMIRSCYSGRVSAAFHANSSEPALLFRNIKCNYVFNNSLTRQLQPINYFDSYLGCVVNAYNSTAISVQTCDLTVGSGYCVDYSKNRRSRGAITTGYRFTNFEPFTVNSVNDSLEPVGGLYEIQIPSEFTIGNMVEFIQTSSPKVTIDCAAFVCGDYAACKSQLVEYGSFCDNINAILTEVNELLDTTQLQVANSLMNGVTLSTKLKDGVNFNVDDINFSPVLGCLGSECSKASSRSAIEDLLFDKVKLSDVGFVEAYNNCTGGAEIRDLICVQSYKGIKVLPPLLSENQISGYTLAATSASLFPPWTAAAGVPFYLNVQYRINGLGVTMDVLSQNQKLIANAFNNALYAIQEGFDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFSYVPTKYVTARVSPGLCIAGDRGIAPKSGYFVNVNNTWMYTGSGYYYPEPITENNVVVMSTCAVNYTKAPYVMLNTSIPNLPDFKEELDQWFKNQTSVAPDLSLDYINVTFLDLQVEMNRLQEAIKVLNQSYINLKDIGTYEYYVKWPWYVWLLICLAGVAMLVLLFFICCCTGCGTSCFKKCGGCCDDYTGYQELVIKTSHDD

SEQ ID NO: 15 - DNA, 4071 bp, coronavirus
atgttattaattatttttattttgcctacaacattagctgttataggtgattttaattgtactaattttgctattaatgatttaaacaccacagttcctcgcataagtgagtatgttgtggatgtttcttatggtttgggtacatattatatacttgatcgtgtttatttaaatactactatattatttactggttatttccctaaatctggtgccaattttagggatctatctttaaaaggtactacatatttgagtactctttggtatcagaaaccctttttatctgattttaataatggtattttttctagagttaagaatactaagttgtatgttaataaaactttgtatagtgagtttagtactatagttataggtagtgtttttattaacaactcttatactattgttgttcaacctcataatggtgttttggagattacagcttgtcaatacactatgtgtgagtatcctcatactatttgtaaatctaaaggtagttctcgtaatgaatcttggcattttgataaatctgaacctttgtgtctgttcaagaaaaattttacttataatgtttctacagattggttgtattttcatttttatcaagaacgtggcactttttatgcttattatgctgattctggcatgcctactacttttttatttagtttgtatcttggtactcttttatctcattattatgttttgcctttgacttgtaatgctatatcttctaatactgataatgagactttacaatattgggtcacacctttgtctaaacgccaatatcttcttaaatttgacaaccgtggtgttattactaatgctgttgattgttctagtagtttctttagcgagattcaatgtaaaactaaatctttattacctaatactggtgtttatgacttatctggttttactgttaagcctgttgcaactgtacatcgtcgtattcctgatttacctgattgtgacattgataaatggcttaacaattttaatgtaccctcacctcttaattgggaacgtaaaattttttctaattgcaactttaatttgagtactttgcttcgtttagttcatactgattctttttcttgtaataattttgatgaatctaagatatatggtagttgttttaagagtattgttttagataaatttgccatacccaactccagacgatctgatttgcagttgggcagttctggttttctgcaatcttctaattataaaattgacactacttctagttcttgtcaattgtattatagtttgcctgcaattaatgttactattaataattataatccttcttcttggaatagaaggtatggttttaataattttaatttgagctctcatagtgttgtttactcacgttattgtttttctgttaataatactttttgtccttgtgctaaaccttcttttgcttcaagttgcaagagtcataaaccaccttctgcttcctgtcctattggtactaattatcgttcttgtgagagtactactgtactcgaccacactgactggtgtaggtgttcttgtttacctgatcctataactgcttatgaccctaggtcttgttctcaaaaaaagtctctggttggtgttggtgaacattgtgcagggttcggtgttgatgaagaaaagtgtggtgtattggatggatcatataatgtttcttgtctttgtagtactgatgcctttctaggttggtcttatgacacttgcgtcagtaacaaccgttgtaatattttttctaattttattttaaatggtatcaatagtggtaccacttgttctaatgatttattgcagcctaatactgaagtttttactgatgtttgtgttgattacgacctttatggtattacaggacaaggtatttttaaagaagtttctgctgtttattataatagttggcaaaatcttttgtatgattctaatggcaacattattggttttaaagattttgttactaataaaacatataatattttcccttgttatgcaggaagagtttctgctgcttttcatcaaaatgcttcctctttggctttactttatcgtaatttaaaatgtagctatgttttgaataatatttctttaactactcagccatattttgatagttatcttggttgcgtttttaatgctgataatttaactgattattctgtttcttcttgtgctcttcgcatgggtagtggtttttgtgttgattataactcaccttcttcttcctcttcgcgtcgtaaacgtagaagtatttctgcttcttatcgttttgttacttttgaaccctttaatgtcagttttgttaatgacagtattgagtctgtgggtggtctttatgagatcaaaattcccactaactttactatagttggtcaagaggaatttattcaaactaattctcctaaagttactattgattgttctttatttgtctgttctaattatgcagcttgccatgacttattgtcagagtatggcactttttgtgataatattaatagtattttagatgaagttaatggtttacttgatactactcaattgcatgtagctgatactcttatgcaaggtgtcacacttagctccaatcttaatactaatttgcattttgatgttgataatattaattttaaatccctagttggatgtttaggtccacactgcggttcttcttctcgttctttttttgaagatttattgtttgacaaagttaaactttcagatgttggttttgttgaagcttataacaattgtactggtggtagtgaaattagagatcttctttgtgtacaatcctttaatggtattaaagttttgcctcctattttgtctgaatctcaaatttctggttacaccacagccgctactgttgctgctatgtttccaccatggtcagcagcagctggcataccattttctcttaatgtacaatatagaattaatggtttgggtgttactatggatgttcttaataaaaatcaaaagttgatagctactgcttttaataatgctcttctttctattcagaatggttttagtgctaccaactctgcacttgctaaaatacaaagtgttgttaattctaatgctcaagcacttaatagtttgttacagcaattatttaataaatttggtgcaattagttcttctttacaagaaattttatctcgtctcgatgctttagaggctcaggttcagattgataggcttattaatggtcgtttaactgctttaaatgcttatgtctctcaacagcttagtgatatttctcttgtaaaatttggtgctgctttagctatggagaaggttaatgagtgtgttaaaagtcaatctcctcgtattaatttttgtggtaatggtaatcatattttgtcattagttcaaaatgctccttatggtttgttgtttatgcattttagttataaacctatttcttttaaaactgttttagtaagtcctggtttgtgtatatcaggtgatgtaggtattgcacctaaacaagggtattttattaaacataatgatcattggatgttcactggtagttcttactattatcctgaaccaatttcagataaaaatgttgtttttatgaatacttgttctgttaattttactaaagcgcctcttgtttatttgaatcattctgtaccaaaattgtctgattttgaatctgagttatctcattggtttaaaaatcaaacatccattgcgcctaatttgactttaaatcttcatactattaatgctacttttttagatttgtattatgagatgaatcttattcaagagtctattaagtctttgaataatagttatatcaatcttaaagatataggtacatatgaaatgtatgtaaaatggccttggtatgtttggctactaatttctttttcatttataatattccttgtattgctcttttttatatgttgttgtactggttgtggttctgcatgttttagtaaatgtcataattgttgtgatgagtatggtggtcatcatgattttgttatcaaaacatctcatgatgattag

SEQ ID NO: 16 - protein, 1356 aa, coronavirus
MLLIIFILPTTLAVIGDFNCTNFAINDLNTTVPRISEYVVDVSYGLGTYYILDRVYLNTTILFTGYFPKSGANFRDLSLKGTTYLSTLWYQKPFLSDFNNGIFSRVKNTKLYVNKTLYSEFSTIVIGSVFINNSYTIVVQPHNGVLEITACQYTMCEYPHTICKSKGSSRNESWHFDKSEPLCLFKKNFTYNVSTDWLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSLLPNTGVYDLSGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVFTDVCVDYDLYGITGQGIFKEVSAVYYNSWQNLLYDSNGNIIGFKDFVTNKTYNIFPCYAGRVSAAFHQNASSLALLYRNLKCSYVLNNISLTTQPYFDSYLGCVFNADNLTDYSVSSCALRMGSGFCVDYNSPSSSSSRRKRRSISASYRFVTFEPFNVSFVNDSIESVGGLYEIKIPTNFTIVGQEEFIQTNSPKVTIDCSLFVCSNYAACHDLLSEYGTFCDNINSILDEVNGLLDTTQLHVADTLMQGVTLSSNLNTNLHFDVDNINFKSLVGCLGPHCGSSSRSFFEDLLFDKVKLSDVGFVEAYNNCTGGSEIRDLLCVQSFNGIKVLPPILSESQISGYTTAATVAAMFPPWSAAAGIPFSLNVQYRINGLGVTMDVLNKNQKLIATAFNNALLSIQNGFSATNSALAKIQSVVNSNAQALNSLLQQLFNKFGAISSSLQEILSRLDALEAQVQIDRLINGRLTALNAYVSQQLSDISLVKFGAALAMEKVNECVKSQSPRINFCGNGNHILSLVQNAPYGLLFMHFSYKPISFKTVLVSPGLCISGDVGIAPKQGYFIKHNDHWMFTGSSYYYPEPISDKNVVFMNTCSVNFTKAPLVYLNHSVPKLSDFESELSHWFKNQTSIAPNLTLNLHTINATFLDLYYEMNLIQESIKSLNNSYINLKDIGTYEMYVKWPWYVWLLISFSFIIFLVLLFFICCCTGCGSACFSKCHNCCDEYGGHHDFVIKTSHDD

SEQ ID NO: 17 - DNA, 4071 bp, coronavirus
atgaaacttttcttgattttgcttgttttgcccctggcctcttgctttttcacatgtaatagtaatgctaatctctctatgttacaattaggtgttcctgacaattcttcaactattgttacgggtttattgccaactcattggttttgtgctaatcagagtacatctgtttactcagccaatggtttcttttatattgatgttggtaatcaccgtagtgcttttgcgctccatactggttattatgatgctaatcagtattatatttatgttactaatgaaataggcttaaatgcttctgttactcttaagatttgtaagtttagtagaaacactacttttgattttttaagtaatgcttctagttcttttgactgtatagttaatttgttatttacagaacagttaggtgcgcctttgggcataactatatctggtgaaactgtgcgtctgcatttatataatgtaactcgtactttttatgtgccagcagcttataaacttactaaacttagtgttaaatgttactttaactattcctgtgtttttagtgttgtcaacgccaccgttactgtgaatgtcaccacacataatggccgtgtagttaactacactgtttgtgatgattgtaatggttatactgataacatattttctgttcaacaggatggccgcattcctaatggtttcccttttaataattggtttttgttaactaatggttccacactagtggacggggtctctagactttatcaaccactccgtttaacttgtttatggcctgtacctggtcttaaatcttcaactggttttgtttattttaatgccactggttctgatgttaattgtaacggctatcaacataattctgttgttgatgttatgcgttacaatcttaacttcagtgctaattctttggacaatctcaagagtggtgttatagtttttaaaactttacagtacgatgttttgttttattgtagtaattcttcctcaggtgttcttgacaccacaataccttttggcccgtcctctcaaccttattactgttttataaacagcactatcaacactactcatgttagcacttttgtgggtattttaccacccactgtgcgtgaaattgttgttgctagaactggccagttttatattaatggttttaagtatttcgatttgggtttcatagaagctgtcaattttaatgtcacgactgctagcgccacagatttttggacggttgcatttgctacttttgttgatgttttggttaatgttagtgcaactaacattcaaaacttactttattgcgattctccatttgaaaagttgcagtgtgagcacttgcagtttggattgcaggatggtttttattctgcaaattttcttgatgataatgttttgcctgagacttatgttgcactccccatttattatcaacacacggacataaattttactgcaactgcatcttttggtggttcttgttatgtttgtaaaccacaccaggttaatatatctcttaatggtaacacttcagtgtgtgttagaacatctcatttttcaattaggtatatttataaccgcgttaagagtggttcaccaggtgactcttcatggcacatttatttaaagagtggcacttgtccattttctttttctaagttaaataattttcaaaagttcaagactatttgtttctcaaccgtcgaagtgcctggtagttgtaattttccgcttgaagccacctggcattacacttcttatactattgttggtgctttgtatgttacttggtctgaaggtaattctattactggtgtaccttatcctgtctctggtattcgtgagtttagtaatttagttttaaataattgtaccaaatataatatttatgattatgttggtactggaattatacgttcttcaaaccagtcacttgctggtggtattacatatgtttctaactctggtaatttacttggttttaaaaatgtttccactggtaacatttttattgtgacaccatgtaaccaaccagaccaagtagctgtttatcaacaaagcattattggtgccatgaccgctgttaatgagtctagatatggcttgcaaaacttactacagttacctaacttttattatgttagtaatggtggtaacaattgcactacggccgttatgacttattctaattttggtatttgtgctgatggttctttgattcctgttcgtccgcgtaattctagtgataatggtatttcagccataatcactgctaatttatccattccttctaactggactacttcagttcaagttgagtacctccaaattactagtactccaatagttgttgattgtgctacttatgtgtgtaatggtaaccctcgctgtaagaatctacttaagcagtatacttctgcttgtaaaactattgaagatgccttacgacttagtgctcatttggaaactaatgatgttagtagtatgctaactttcgatagcaatgcttttagtttggctaatgttactagttttggagattataacctttctagtgttttacctcagagaaacattcgttcaagccgtatagcaggacgtagtgctttggaagatttgttgtttagcaaagttgttacatctggtttgggtactgttgatgttgactataagtcttgtactaaaggtctttctattgctgaccttgcttgtgctcagtactacaatggcataatggttttgccaggtgttgctgatgctgaacgtatggccatgtacacaggttctcttataggtggcatggtgctcggaggtcttacatcagcagccgccatacctttttctttggcactgcaagcacgacttaactatgttgctttacaaactgatgtgcttcaagaaaatcagaaaattttggctgcatcatttaataaggctattaataatattgttgcttcttttagtagcgttaatgatgctattacacaaactgcagaggctatacatactgttactattgcacttaataagattcaggatgttgttaatcaacagggtagtgctcttaaccatctcacttcacaattgagacataattttcaggccatttctaattcaattcaggctatttatgaccggcttgattcaattcaagccgatcaacaagttgacagattaattactggacggcttgcagctttgaatgcatttgtttcccaagttttgaataaatatactgaagttcgtggttcaagacgcttagcacagcagaagattaatgaatgtgtcaagtcacaatctaatagatatggtttttgtggcaatggcactcacatcttttcaatcgtcaactcagctccagatggtttgctttttcttcatactgttttgctgccaactgattacaagaatgtaaaggcgtggtctggtatctgtgttgatggcatttatggctatgttctgcgtcaacctaacttggttctttattctgataatggtgtctttcgtgtaacttccagggtcatgtttcaacctcgcttacctgttttgtctgattttgtgcaaatatataattgtaatgttacttttgttaacatatctcgtgttgagttacatactgtcatacctgactacgttgatgttaataaaacattacaagagtttgcacaaaacttaccaaagtatgttaagcctaattttgacttgactccttttaatttaacatatcttaatttgagttctgagttgaagcaactcgaagctaaaactgctagtctttttcaaactactgttgaattacaaggtcttattgatcagattaacagtacatatgttgatttgaagttgcttaataggtttgaaaattatatcaaatggccttggtgggtttggctcattatttctgttgtttttgttgtattgttgagtcttcttgtgttttgttgtctttctacaggttgttgtggttgttgcaattgtttaacttcatcaatgcgaggctgttgtgattgtggttcaactaaacttccttattacgaatttgaaaaggtccacgttcaataa

SEQ ID NO: 18 - protein, 1356 aa, coronavirus
MKLFLILLVLPLASCFFTCNSNANLSMLQLGVPDNSSTIVTGLLPTHWFCANQSTSVYSANGFFYIDVGNHRSAFALHTGYYDANQYYIYVTNEIGLNASVTLKICKFSRNTTFDFLSNASSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVVDVMRYNLNFSANSLDNLKSGVIVFKTLQYDVLFYCSNSSSGVLDTTIPFGPSSQPYYCFINSTINTTHVSTFVGILPPTVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDSIQADQQVDRLITGRLAALNAFVSQVLNKYTEVRGSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENYIKWPWWVWLIISVVFVVLLSLLVFCCLSTGCCGCCNCLTSSMRGCCDCGSTKLPYYEFEKVHVQ

SEQ ID NO: 19 - DNA, 3522 bp, coronavirus
atgtttgttttgcttgttgcatatgccttgttgcatattgctggttgtcaaactacaaatgggctgaacactagttactctgtttgcaacggctgtgttggttattcagaaaatgtatttgctgttgagagtggtggttatataccctccgactttgcattcaataattggttccttctaactaatacctcatctgttgtagatggtgttgtgaggagttttcagcctttgttgcttaattgcttatggtctgtttctggcttgcggtttactactggttttgtctattttaatggtactgggagaggtgattgtaaaggtttttcctcagatgttttgtctgatgtcatacgttacaacctcaattttgaagaaaaccttagacgtggaaccattttgtttaaaacatcttatggtgttgttgtgttttattgtaccaacaacactttagtttcaggtgatgctcacataccatttggtacagttttgggcaatttttattgctttgtaaatactactattggcaatgaaactacgtctgcttttgtgggtgcactacctaagacagttcgtgagtttgttatttcacgcacaggacatttttatattaatggctatcgctatttcactttaggtaatgtagaagccgttaatttcaatgtcactactgcagaaaccactgatttttgtactgttgcgttagcttcttatgctgacgttttggttaatgtgtcacaaacctctattgctaatataatttattgcaactctgttattaacagactgagatgtgaccagttgtcctttgatgtaccagatggtttttattctacaagccctattcaatccgttgagctacctgtgtctattgtgtcgctacctgtttatcataaacatacgtttattgtgttgtacgttgacttcaaacctcagagtggcggtggcaagtgctttaactgttatcctgctggtgttaatattacactggccaattttaatgaaactaaagggcctttgtgtgttgacacatcacacttcactaccaaatacgttgctgtttatgccaatgttggtaggtggagtgctagtattaacacgggaaattgccctttttcttttggcaaagttaataactttgttaaatttggcagtgtatgtttttcgctaaaggatatacccggtggttgcgcaatgcctatagtggctaattgggcttatagtaagtactatactataggctcattgtatgtttcttggagtgatggtgatggaattactggcgtcccacaacctgttgagggtgttagttcctttatgaatgttacattggacaaatgtactaaatataatatttatgatgtatctggtgtgggtgttattcgcgttagcaatgacacctttcttaatggaattacgtacacatcaacttcaggtaaccttctgggttttaaagatgttactaagggcaccatctactctatcactccttgtaacccaccagatcagcttgttgtttatcagcaagctgttgttggtgctatgttgtctgaaaattttactagttacggcttttctaatgttgtagaactgccgaaatttttctatgcgtccaatggcacttataattgcacagacgctgttttaacttattctagttttggcgtttgtgcagatggttctataattgctgttcaaccacgtaatgtttcatatgatagtgtttcagctatcgtcacagctaatttgtctataccttccaattggaccacttcggtccaggttgagtatttacaaattacaagtacacctatcgtagttgattgctccacttatgtttgcaatggtaatgtgcgctgtgttgaattgcttaagcagtatacttctgcttgtaaaactattgaagacgccttaagaaatagcgccaggctggagtctgcagatgttagtgagatgctcacttttgacaagaaagcgtttacacttgctaatgttagtagttttggtgactacaaccttagcagcgtcatacctagcttgcccacaagtggtagtagagtggctggtcgcagtgccatagaagacatactttttagcaaacttgttacttctggacttggcactgtggacgcagactacaaaaagtgcactaagggtctttccattgctgacttggcttgtgctcaatattataatggcattatggttttgcctggcgtcgctgatgctgaacgaatggccatgtatacaggttctttaattggtggaattgctttaggaggtctaacatcagccgtttcaataccattttcattagcaattcaggcacgtttaaattatgttgcattgcagactgatgttttacaagaaaatcagaaaattcttgctgcatcttttaacaaagcaatgaccaacatagtagatgcctttactggtgttaatgatgctattacacaaacttcacaagccctacaaacagttgctactgcacttaacaagatccaggatgttgttaatcaacaaggcaactcattgaaccatttaacttctcagttgaggcagaattttcaagctatctctagctctattcaggctatctatgacagacttgacactattcaggctgatcaacaagtagataggctgattactggtagattggctgctttgaatgtattcgtttctcatacattgactaagtacactgaagttcgtgcttccagacagcttgcacaacaaaaagtgaatgagtgtgtcaaatcccagtctaagcgttatggcttctgtggaaatggcactcacattttctcaattgttaatgctgctcctgaggggcttgtttttctccacactgtcttgttgccgacacaatataaggatgttgaagcgtggtctgggttgtgcgttgatggtacaaacggttatgtgttgcgacaacctaatcttgctctttacaaagaaggcaattattatagaatcacatctcgcataatgtttgaaccacgtattcctaccatggcagattttgttcaaattgaaaattgcaatgtcacatttgttaacatttctcgctctgagttgcaaaccattgtgccagagtatattgatgttaataagacgctgcaagaattaagttacaaattgccaaattacactgttccagacctagttgtcgaacagtacaaccagactattttgaatttgaccagtgaaattagcacccttgaaaataaatctgcggagcttaattacactgttcaaaaattgcaaactctgattgacaacataaatagcacattagtcgacttaaagtggctcaaccgggttgagacttacatcaagtggccgtggtgggtgtggttgtgcatttcagtcgtgctcatctttgtggtgagtatgttgctattatgttgttgttctactggttgctgtggcttctttagttgttttgcatcttctattagaggttgttgtgaatcaactaaacttccttattacgacgttgaaaagatccacatacagtaa

SEQ ID NO: 20 - protein, 1173 aa, coronavirus
MFVLLVAYALLHIAGCQTTNGLNTSYSVCNGCVGYSENVFAVESGGYIPSDFAFNNWFLLTNTSSVVDGVVRSFQPLLLNCLWSVSGLRFTTGFVYFNGTGRGDCKGFSSDVLSDVIRYNLNFEENLRRGTILFKTSYGVVVFYCTNNTLVSGDAHIPFGTVLGNFYCFVNTTIGNETTSAFVGALPKTVREFVISRTGHFYINGYRYFTLGNVEAVNFNVTTAETTDFCTVALASYADVLVNVSQTSIANIIYCNSVINRLRCDQLSFDVPDGFYSTSPIQSVELPVSIVSLPVYHKHTFIVLYVDFKPQSGGGKCFNCYPAGVNITLANFNETKGPLCVDTSHFTTKYVAVYANVGRWSASINTGNCPFSFGKVNNFVKFGSVCFSLKDIPGGCAMPIVANWAYSKYYTIGSLYVSWSDGDGITGVPQPVEGVSSFMNVTLDKCTKYNIYDVSGVGVIRVSNDTFLNGITYTSTSGNLLGFKDVTKGTIYSITPCNPPDQLVVYQQAVVGAMLSENFTSYGFSNVVELPKFFYASNGTYNCTDAVLTYSSFGVCADGSIIAVQPRNVSYDSVSAIVTANLSIPSNWTTSVQVEYLQITSTPIVVDCSTYVCNGNVRCVELLKQYTSACKTIEDALRNSARLESADVSEMLTFDKKAFTLANVSSFGDYNLSSVIPSLPTSGSRVAGRSAIEDILFSKLVTSGLGTVDADYKKCTKGLSIADLACAQYYNGIMVLPGVADAERMAMYTGSLIGGIALGGLTSAVSIPFSLAIQARLNYVALQTDVLQENQKILAASFNKAMTNIVDAFTGVNDAITQTSQALQTVATALNKIQDVVNQQGNSLNHLTSQLRQNFQAISSSIQAIYDRLDTIQADQQVDRLITGRLAALNVFVSHTLTKYTEVRASRQLAQQKVNECVKSQSKRYGFCGNGTHIFSIVNAAPEGLVFLHTVLLPTQYKDVEAWSGLCVDGTNGYVLRQPNLALYKEGNYYRITSRIMFEPRIPTMADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKWPWWVWLCISVVLIFVVSMLLLCCCSTGCCGFFSCFASSIRGCCESTKLPYYDVEKIHIQ

SEQ ID NO: 21 - DNA, 3822 bp, coronavirus
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 22 - protein, 1273 aa, coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - protein, 1270 aa, coronavirus
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 24 - protein, 1270 aa, coronavirus
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - protein, 1270 aa, coronavirus
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQPYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 26 - protein, 1273 aa, coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - protein, 1273 aa, coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 28 - protein, 1273 aa, coronavirus; identical to SEQ ID NO: 26

SEQ ID NO: 29 - protein, 1273 aa, coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 30 - protein, 1273 aa, coronavirus
MFVFLVLLPLVSIQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 31 - protein, 6 aa, coronavirus
GQGVPI

SEQ ID NO: 32 - protein, 6 aa, coronavirus
RNLVPI

SEQ ID NO: 33 - protein, 12 aa, coronavirus
PRWYFYYLGTGP

SEQ ID NO: 34 - protein, 12 aa, coronavirus
PKVHFYYLGTGP

SEQ ID NO: 35 - protein, 12 aa, coronavirus
PKLHFYYLGTGP

SEQ ID NO: 36 - protein, 6 aa, coronavirus
KPRQKR

SEQ ID NO: 37 - protein, 6 aa, coronavirus
KPRWKR

SEQ ID NO: 38 - protein, 9 aa, coronavirus
RSAIEDLLF

SEQ ID NO: 39 - protein, 9 aa, coronavirus
RSFIEDLLF

SEQ ID NO: 40 - protein, 9 aa, coronavirus
RSFFEDLLF

SEQ ID NO: 41 - protein, 6 aa, coronavirus
VLPPLL

SEQ ID NO: 42 - protein, 6 aa, coronavirus
VLPPIL

SEQ ID NO: 43 - protein, 6 aa, coronavirus
NQKLIA

SEQ ID NO: 44 - protein, 6 aa, coronavirus
ILSRLD

SEQ ID NO: 45 - protein, 6 aa, coronavirus
QIDRLI

SEQ ID NO: 46 - protein, 5 aa, coronavirus
KWPWY

SEQ ID NO: 47 - protein, 10 aa, coronavirus
KVNECVKSQS

SEQ ID NO: 48 - protein, 5 aa, coronavirus
FCGNG

SEQ ID NO: 49 - protein, 8 aa, coronavirus
KWPWYVWL

SEQ ID NO: 50 - protein, 8 aa, coronavirus
KWPWWVWL

SEQ ID NO: 51 - protein, 395 aa, Artificial sequence
  synthetic sequence
MSDNGPQNQRNAPRITFGGPSDSTGSNQNGERSGARSKQRRPQGLPNNTASWFTALTQHGKEDLKFPRNTNSSPDDQIGYYRRATRRIRGGDGKMKDLSEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 52 - DNA, 554 bp, Artificial sequence
  synthetic sequence
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagc

SEQ ID NO: 53 - protein, 218 aa, Artificial sequence
  synthetic sequence
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 54 - DNA, 18 bp, Artificial sequence
  synthetic sequence
catcaccatcaccatcac

SEQ ID NO: 55 - protein, 6 aa, Artificial sequence
  synthetic sequence
HHHHHH

SEQ ID NO: 56 - protein, 8 aa, Artificial sequence
  synthetic sequence
DYKDDDDK

SEQ ID NO: 57 - protein, 9 aa, Artificial sequence
  synthetic sequence
YPYDYPDYA

SEQ ID NO: 58 - protein, 10 aa, Artificial sequence
  synthetic sequence
EQKLISEEDL

SEQ ID NO: 59 - protein, 13 aa, Artificial sequence
  synthetic sequence
SRLEEELRRRLTE

SEQ ID NO: 60 - protein, 14 aa, Artificial sequence
  synthetic sequence
GKPIPNPLLGLDST

SEQ ID NO: 61 - protein, 12 aa, Artificial sequence
  synthetic sequence
PDRVRAVSHWSS

SEQ ID NO: 62 - protein, 11 aa, Artificial sequence
  synthetic sequence
MASMTGGQQMG

SEQ ID NO: 63 - protein, 18 aa, Artificial sequence
  synthetic sequence
TKENPRSNQEFSYDDNES

(19) United States
(12) Patent Application Publication
(10) Pub. No.: US 2023/0168247 A1
(43) Pub. Date: Jun. 1, 2023

(54) SPECIFICITY ENHANCING REAGENTS FOR COVID-19 ANTIBODY TESTING

(71) Applicant: University of Virginia Patent Foundation, Charlottesville, VA (US)

(72) Inventors: James C. Zimring, Charlottesville, VA (US); Lindsay Ann Legendre Bazydlo, Charlottesville, VA (US); Joesph Richard Wiencek, Charlottesville, VA (US); James David Gorham, Charlottesville, VA (US); Heather Howie, Charlottesville, VA (US)

(73) Assignee: University of Virginia Patent Foundation, Charlottesville (US)

(21) Appl. No.: 17/921,039

(22) PCT Filed: Apr. 26, 2021
(86) PCT No.: PCT/US2021/029187

§ 371 Date: Oct. 24, 2022

Related U.S. Application Data
(60) Provisional application No. 63/015,215, filed on Apr. 24, 2020.

(51) Int. Cl. G01N 33/543; G01N 33/68

(52) U.S. Cl. CPC G01N 33/54393; G01N 33/6854; G01N 2469/20

(57) ABSTRACT

Methods of detecting SARS-CoV-2 antibodies with improved specificity are described. The methods can include contacting a sample potentially containing SARS-CoV-2 antibodies with a reagent to remove non-anti-SARS-CoV-2 antibodies, such as a reagent comprising an epitope from a nucleocapsid protein or spike protein of a common coronavirus. The methods can include contacting the sample with a mutant SARS-CoV-2 nucleocapsid protein or spike protein comprising a reduced number of common coronavirus epitopes. The methods can involve comparing results from an immunoassay performed with a SARS-CoV-2 protein to results obtained from immunoassays performed with analogous protein from one or more common coronaviruses.

### CROSS REFERENCE TO RELATED APPLICATIONS

This application claims benefit of U.S. Provisional Application Ser. No. 63/015,215, filed Apr. 24, 2020, the disclosure of which is incorporated herein by reference in its entirety.

### TECHNICAL FIELD

The presently disclosed subject matter is directed to methods of detecting the presence of antibodies for severe acute respiratory syndrome coronavirus 2 (SARS-CoV-2), the causative agent for Coronavirus disease 2019 (COVID-19), and reducing the likelihood of false positives arising from the presence of antibodies to common coronaviruses in a sample.

### BACKGROUND

In the winter of 2019, a novel coronavirus emerged that has been designated severe acute respiratory syndrome coronavirus 2 (SARS-CoV-2), the causative agent of Coronavirus disease 2019 (COVID-19). SARS-CoV-2 has led to a worldwide pandemic leading to widespread infection and death. Managing the epidemic, as well as individual patient care, requires widespread and accurate testing of both active viral infection and post-infection serology. Sensitive and specific RT-PCR based testing of active infection is actively in place; however, such tests are not informative outside of the period of active infection and cannot be used to determine if someone has been previously infected. Thus, after the acute infection phase, assessment of antibodies specific for COVID-19 (i.e., specific for SARS-CoV-2) is used to determine if a person has been previously infected.

It is currently unclear if antibodies are protective, but such determination will be helpful regarding patient care and/or the development of convalescent plasma as a potential therapy. Further, the use of antibody responses to determine if a person has been previously infected is of interest, from a public health standpoint and regarding management of the epidemic (as well as societal maneuvers to mitigate spread). Thus, for multiple reasons, there is an ongoing need for additional methods for the more specific determination of serological response to SARS-CoV-2.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter provides a method of performing an immunoassay to detect a presence or absence of an antibody for severe acute respiratory syndrome coronavirus 2 (SARS-CoV-2) in a sample comprising one or more antibodies, the method comprising: incubating the sample with a cross-reactivity neutralizing reagent (CNR) comprising, consisting essentially of, or consisting of one or more proteins or protein fragments from a common coronavirus, wherein said one or more proteins or protein fragments are selected from the group comprising a spike protein, a spike protein fragment, a nucleocapsid protein, and a nucleocapsid protein fragment, wherein the incubating is performed under conditions sufficient to form an antibody/CNR complex between the CNR and any antibody present in the sample specific for the CNR; incubating the sample with a SARS-CoV-2 protein or a fragment thereof under conditions sufficient to form an antibody/SARS-CoV-2 protein complex between the SARS-CoV-2 protein or fragment thereof and any antibody in the sample specific for the SARS-CoV-2 protein or fragment thereof; treating the sample to remove any antibody/CNR complex present in the sample, thereby forming a subtracted sample; and analyzing the subtracted sample to determine the presence or absence of any antibody/SARS-CoV-2 protein complex, thereby detecting a presence or absence of antibody binding to the SARS-CoV-2 protein or fragment thereof. In some embodiments, the sample is a blood sample or a serum sample.

In some embodiments, the CNR comprises, consists essentially of, or consists of one or more recombinant proteins or protein fragments. In some embodiments, the common coronavirus is selected from the group comprising coronavirus OC43 (OC43-CoV), coronavirus HKU1 (HKU1-CoV), coronavirus NL63 (NL63-CoV), and coronavirus 229E (229E-CoV). In some embodiments, the CNR comprises, consists essentially of, or consists of one or more nucleocapsid proteins or protein fragments. In some embodiments, the CNR comprises, consists essentially of, or consists of one of SEQ ID NOS. 2, 4, 6, 8, 10, 14, 16, 18, and 20.

In some embodiments, the SARS-CoV-2 protein or fragment thereof comprises or consists of one of SEQ ID NOS: 12 and 22-30. In some embodiments, the SARS-CoV-2 protein of fragment thereof is immobilized on a solid support. In some embodiments, the solid support is a microtiter plate.

In some embodiments, the presently disclosed subject matter provides a method of performing an immunoassay to detect a presence or absence of an antibody for SARS-CoV-2 in a sample comprising one or more antibodies, the method comprising: incubating the sample with a mutant protein or protein fragment for SARS-CoV-2 under conditions sufficient to form an antibody/mutant protein complex between the mutant protein or protein fragment for SARS-CoV-2 and an antibody in the sample specific for said mutant protein or protein fragment for SARS-CoV-2, wherein said mutant protein or protein fragment for SARS-CoV-2 comprises or consists of: (i) a common epitope deleted mutant nucleocapsid protein or a fragment thereof, wherein said common epitope deleted mutant nucleocapsid protein is a recombinant protein having an amino acid sequence of a nucleocapsid protein of SARS-CoV-2 wherein one or more common coronavirus nucleocapsid protein epitope has been removed, wherein each of said one or more common coronavirus nucleocapsid protein epitope has an amino acid sequence selected from the group comprising SEQ ID NOS: 31-37; or (ii) a common epitope deleted mutant spike protein or a fragment thereof, wherein said common epitope deleted mutant spike protein is a recombinant protein having an amino acid sequence of a spike protein of SARS-CoV-2 wherein one or more common coronavirus spike protein epitope has been removed, wherein each of said one or more common coronavirus spike protein epitope has an amino acid sequence selected from the group comprising SEQ ID NOS: 38-50; and analyzing the sample to determine the presence or absence of an antibody/mutant protein complex, thereby determining the presence or absence of an antibody in the sample specific for SARS-CoV-2. In some embodiments, the sample is a blood sample or a serum sample.

In some embodiments, the mutant protein or protein fragment for SARS-CoV-2 is a mutant spike protein, wherein the mutant spike protein has an amino acid sequence selected from SEQ ID NOS: 22-30 from which one or more common coronavirus spike protein epitope has been removed. In some embodiments, the mutant protein or protein fragment for SARS-CoV-2 is a mutant nucleocapsid protein, wherein the mutant nucleocapsid protein has an amino acid sequence of SEQ ID NO: 12 from which one or more common coronavirus nucleocapsid protein epitope has been removed. In some embodiments, the one or more common coronavirus nucleocapsid protein epitope is a peptide comprising an amino acid sequence selected from the group comprising GQGVP (SEQ ID NO: 31), PRWYFYYLGTGP (SEQ ID NO: 33), and KPRQKR (SEQ ID NO: 36). In some embodiments, the mutant protein comprises or consists of an amino acid having an amino acid sequence: MSDNGPQNQRNAPRITFGGPSDSTGSNQNGERSGARSKQRRPQGLPNNTAS WFTALTQHGKEDLKFPRNTNSSPDDQIGYYRRATRRIRGGDGKMKDLSEAG LPYGANKDGIIWVATEGALNTPKDHIGTRNPANNAAIVLQLPQGTTLPKGFY AEGSRGGSQASSRSSSRSRNSSRNSTPGSSRGTSPARMAGNGGDAALALLLL DRLNQLESKMSGKGQQQQGQTVTKKSAAEASKTATKAYNVTQAFGRRGPE QTQGNFGDQELIRQGTDYKHWPQIAQFAPSASAFFGMSRIGMEVTPSGTWL TYTGAIKLDDKDPNFKDQVILLNKHIDAYKTFPPTEPKKDKKKKADETQALP QRQKKQQTVTLLPAADLDDFSKQLQQSMSSADSTQA (SEQ ID NO: 51), or a fragment thereof, or having an amino acid sequence having 95% homology to said amino acid sequence or a fragment thereof.

In some embodiments, the presently disclosed subject matter provides a mutant protein comprising, consisting essentially of, or consisting of the amino acid of SEQ ID NO: 51. In some embodiments, the mutant protein further comprises a tag. In some embodiments, the tag is selected from the group comprising a glutathione-S-transferase (GST) tag, a His tag, a FLAG tag, a hemagglutinin (HA) tag, a cMyc tag, an ALFA-tag, a V5-tag, a Spot-tag, a T7-tag, an NE tag, and combinations thereof.

In some embodiments, the presently disclosed subject matter provides a kit for performing an immunoassay comprising the mutant protein of claim 16, wherein said mutant protein is immobilized on a solid support. In some embodiments, the kit further comprises a detection reagent, wherein the detection reagent comprises a labeled reporter antibody that binds to a constant region of an antibody.

In some embodiments, the presently disclosed subject matter provides a method of performing an immunoassay to detect a presence or absence of an antibody for SARS-CoV-2 in a sample comprising one or more antibodies, the method comprising: receiving a sample from a patient suspected of having been exposed to SARS-CoV-2; splitting the sample into two to five aliquots; incubating one of the two to five aliquots with a viral protein from SARS-CoV-2 or a fragment thereof under conditions sufficient to form antibody/protein complexes between the viral protein or fragment thereof and any antibody in the sample specific for the viral protein; incubating each remaining aliquot of the two to five aliquots with a corresponding viral protein or fragment thereof from a different common coronavirus selected from the group comprising OC43-CoV, HKU1-CoV, NL63-CoV, and 229E-CoV under conditions sufficient to form antibody/protein complexes between the corresponding viral protein or fragment thereof and any antibody in the sample specific for the corresponding viral protein; determining a signal associated with antibody binding for each of the two to five aliquots, thereby determining a plurality of binding signals for the sample, wherein each of the plurality of binding signals is for a different viral protein; and comparing the binding signals, thereby detecting the presence or absence of an antibody to SARS-CoV-2. In some embodiments, the viral protein from SARS-CoV-2 and each corresponding viral protein is a spike protein or wherein each viral protein from SARS-CoV-2 and each corresponding viral protein is a nucleocapsid protein. In some embodiments, splitting the sample into two to five aliquots comprises splitting the sample into five aliquots.

Accordingly, it is an object of the presently disclosed subject matter to provide methods of performing immunoassays for detecting the presence or absence of an antibody for SARS-CoV-2. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description and FIGURES.

### BRIEF DESCRIPTION OF THE FIGURES

The presently disclosed subject matter can be better understood by referring to the following FIGURES. The components in the figures are not necessarily to scale, emphasis instead being placed upon illustrating the principles of the presently disclosed subject matter (often schematically). In the FIGURES, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIG. 1A is a schematic diagram showing an immunoassay for the detection of SARS-CoV-2 antibodies from a sample comprising antibodies to both SARS-CoV-2 and common coronaviruses that provides a true positive result through the use of a capture antigen with unique epitopes for SARS-CoV-2.

FIG. 1B is a schematic diagram showing an immunoassay for the detection of SARS-CoV-2 antibodies using a sample comprising only antibodies to common coronaviruses and resulting in a false positive result.

FIG. 1C is a schematic diagram showing an immunoassay for the detection of SARS-CoV-2 antibodies where the use of a cross reactivity neutralizing reagent (CNR) based on epitopes from common coronaviruses results in a true negative result with a sample comprising antibodies to common coronaviruses but no SARS-CoV-2 antibodies.

FIG. 1D is a schematic diagram showing an immunoassay for the detection of SARS-CoV-2 antibodies where use of a cross reactivity neutralizing reagent (CNR) based on epitopes from common coronaviruses results in a true positive result with a sample comprising antibodies to both common coronaviruses and SARS-CoV-2.

### REFERENCE TO SEQUENCE LISTING

The Sequence Listing associated with the instant disclosure has been submitted electronically herewith as an 208 kilobyte file with File Name (3062-127-PCT.ST25.txt), Creation Date (Apr. 26, 2021), Computer System (IBM-PC/MS-DOS/MS-Windows), and Docket No. (3062/127 PCT). The Sequence Listing submitted electronically herewith is hereby incorporated by reference into the instant disclosure.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

In accordance with the presently disclosed subject matter there can be employed conventional chemical, cellular, histochemical, biochemical, molecular biology, microbiology, recombinant DNA, and clinical techniques which are known to those of skill in the art. Such techniques are explained fully in the literature. See for example, Sambrook et al. (1989); Glover (1985); Gait (1984); Harlow & Lane, 1988; Roe et al. (1996); and Ausubel et al. (1995).

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In some embodiments, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, amino acids are represented by the full name thereof, by the three-letter code corresponding thereto, and/or by the one-letter code corresponding thereto, as summarized in Table 1:

TABLE 1
Amino Acids and Codes and Codons Therefor

| | | | Functionally |
|---|---|---|---|
| | 3-Letter | 1-Letter | Equivalent |
| Full Name | Code | Code | Codons |
| Aspartic Acid | Asp | D | GAC GAU |
| Glutamic Acid | Glu | E | GAA GAG |
| Lysine | Lys | K | AAA AAG |
| Arginine | Arg | R | AGA AGG CGA CGC |
| | | | CGG CGU |
| Histidine | His | H | CAC CAU |
| Tyrosine | Tyr | Y | UAC UAU |
| Cysteine | Cys | C | UGC UGU |
| Asparagine | Asn | N | AAC AAU |
| Glutamine | Gln | Q | CAA CAG |
| Serine | Ser | S | ACG AGU UCA UCC |
| | | | UCG UCU |
| Threonine | Thr | T | ACA ACC ACG ACU |
| Glycine | Gly | G | GGA GGC GGG GGU |
| Alanine | Ala | A | GCA GCC GCG GCU |
| Valine | Val | V | GUA GUC GUG GUU |
| Leucine | Leu | L | UUA UUG CUA CUC |
| | | | CUG CUU |
| Isoleucine | Ile | I | AUA AUC AUU |
| Methionine | Met | M | AUG |
| Proline | Pro | P | CCA CCC CCG CCU |
| Phenylalanine | Phe | F | UUC UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

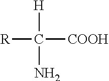

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The terms “sample” and “biological sample”, as used herein, refer to samples obtained from a subject, including, but not limited to, skin, hair, tissue, blood, plasma, cells, sweat and urine. In some embodiments, the sample is sample of a bodily fluid, such as a nasal swab, nasal aspirate, a pharyngeal swab, a respiratory secretion, sweat, urine, a cell or tissue homogenate, a serum sample, a plasma sample, a whole blood sample, or a saliva sample.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

As used herein, the term “conservative amino acid substitution” is defined herein as an amino acid exchange within one of the five groups summarized in Table 2.

TABLE 2
Conservative Amino Acid Substitutions

| Group | Characteristics | Amino Acids |
|---|---|---|
| A. | Small aliphatic, nonpolar or slightly polar | Ala, Ser, Thr, Pro, |
| | residues | Gly |
| B. | Polar, negatively charged residues and their | Asp, Asn, Glu, Gln |
| | amides | |
| C. | Polar, positively charged residues | His, Arg, Lys |
| D. | Large, aliphatic, nonpolar residues | Met Leu, Ile, Val, |
| | | Cys |
| E. | Large, aromatic residues | Phe, Tyr, Trp |

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

The term “coronavirus” as used herein refers to a member of a family of positive-sense, single-stranded RNA viruses that are known to cause severe respiratory illness. The viral genome is capped, polyadenylated, and covered with nucleocapsid (N) proteins. The coronavirus virion includes a viral envelope containing type I fusion glycoproteins referred to as the spike (S) protein. Most coronaviruses have a common genome organization with the replicase gene included in the 5′-portion of the genome, and structural genes included in the 3′-portion of the genome. Non-limiting examples of betacoronaviruses include Middle East respiratory syndrome coronavirus (MERS-CoV), Severe Acute Respiratory Syndrome coronavirus (SARS-CoV), Severe Acute Respiratory Syndrome coronavirus 2 (SARS-CoV-2 or COVID-19), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronaviruses is the Swine Delta Coronavirus (SDCV)

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, an “essentially pure” preparation of a particular protein or peptide is a preparation wherein at least about 95%, and in some embodiments at least about 99%, by weight, of the protein or peptide in the preparation is the particular protein or peptide.

The term “epitope” as used herein refers to an antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on a coronavirus S or N protein. Epitopes can be formed from contiguous amino acids or noncontiguous amino acids juxtaposed by tertiary folding of a protein.

A “fragment” or “segment” is a portion of an amino acid sequence, comprising at least one amino acid, or a portion of a nucleic acid sequence comprising at least one nucleotide. The terms “fragment” and “segment” are used interchangeably herein. As used herein, the term “fragment” of the polypeptide of the presently disclosed subject matter encompasses natural or synthetic portions of the full-length protein, which in some embodiments are capable of specific or selective binding to their natural ligand or of performing a function of the protein. Truncations, alternatively spliced version, and indeed combination of any natural or synthetic portions of the full-length protein are encompassed by the term “fragment”.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules.

When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993). This algorithm is incorporated into the NBLAST and XBLAST programs of Altschul et al., 1990a and Altschul et al. 1990b; and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically or selectively binds to a target compound. A ligand (e.g., an antibody or peptide sequence) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a compound that specifically or selectively binds to a ligand.

A ligand or a receptor (e.g., an antibody or peptide sequence) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane 1988 for a description of immunoassay formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “peptide” typically refers to short polypeptides.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. Representative purification techniques are disclosed herein for antibodies and fragments thereof.

The term “recombinant” can refer to a nucleic acid molecule that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

Thus, “recombinant polynucleotide” can refer to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, origin of replication, ribosome-binding site, etc.) as well.

The term “host cell” refers to a cell in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used. A host cell that comprises a recombinant polynucleotide can be referred to as a “recombinant host cell”. A gene which is expressed in a recombinant host cell wherein the gene comprises a recombinant polynucleotide, produces a “recombinant polypeptide”.

In some embodiments, a “recombinant polypeptide” or a “recombinant protein” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “sample”, as used herein, refers in some embodiments to a biological sample from a subject that includes antibodies produced in the subject, including, but not limited to, normal tissue samples, diseased tissue samples, biopsies, blood, plasma, serum, mucus, nasal aspirate, saliva, feces, semen, tears, and urine. A sample can also be any other source of material obtained from a subject which contains cells, tissues, or fluid of interest. A sample can also be obtained from cell or tissue culture.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

The term “standard”, as used herein, refers to something used for comparison. For example, a standard can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

A “subject” of diagnosis or treatment is an animal, including a human. It also includes pets and livestock. As used herein, the term “subject” refers to an individual (e.g., human, animal, or other organism) to be assessed, evaluated, and/or treated by the methods or compositions of the presently disclosed subject matter. Subjects include, but are not limited to, mammals (e.g., murines, simians, equines, bovines, porcines, canines, felines, and the like), and includes humans. As used herein, the terms “subject” and “patient” are used interchangeably, unless otherwise noted.

As used herein, a “subject in need thereof” is a patient, animal, mammal, or human, who will benefit from the method of this presently disclosed subject matter.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference sequence. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2×standard saline citrate (SSC), 0.1% SDS at 50° C.; in some embodiments in 7% (SD S), 0.5 M NaPO4, 1 mM EDTA at 50° C. with washing in 1×SSC, 0.1% SDS at 50° C.; in some embodiments 7% SDS, 0.5 M NaPO4, 1 mM EDTA at 50° C. with washing in 0.5×SSC, 0.1% SDS at 50° C.; and more in some embodiments in 7% SDS, 0.5 MNaPO4, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The use of the phrase “tissue culture dish or plate” refers to any type of vessel which can be used to plate cells for growth or differentiation.

“Tissue” means (1) a group of similar cells united to perform a specific function; (2) a part of an organism consisting of an aggregate of cells having a similar structure and function; or (3) a grouping of cells that are similarly characterized by their structure and function, such as muscle or nerve tissue.

The term to “treat”, as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter can exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), VL (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ can be reduced under mild conditions to break the disulfide linkage in the hinge region, thereby converting the F(ab′)₂ dimer into an Fab₁ monomer. The Fab₁ monomer is essentially a Fab with part of the hinge region. While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all intact antibody molecules.

An “antibody light chain”, as used herein, refers to the smaller of the two types of polypeptide chains present in all intact antibody molecules.

The term “single chain antibody” refers to an antibody wherein the genetic information encoding the functional fragments of the antibody are located in a single contiguous length of DNA.

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates.

### II. GENERAL CONSIDERATIONS

In the field of clinical diagnostics there is a broad category of methods available for determining an expanding list of clinically relevant analytes. One such category is immunoassays, which are currently used to determine the presence or concentration of various analytes in biological samples. Immunoassays utilize specific binding agents to target analytes in fluids, where at least one such binding agent is generally labeled with a label selected from a variety of compounds, including radioisotopes, enzymes and fluorescent or chemiluminescent compounds, that can be measured, for example, by radioactive disintegrations, enzymatic induced color-producing substrates, fluorescent output or inhibition and/or chemiluminescent light output. Such specific binding agents typically include analyte specific antibodies (immunoglobulins) and antibody fragments, receptors, lectins, and genetically or chemically engineered artificial antibodies. Notable immunoassay methods include, for example, radioimmunoassay (RIA), enzyme-linked immunosorbent assay (ELIZA) (Enzyme-Immunoassay, Edward T. Maggio, CRC Press, 1980), fluorescent immunoassay (FIA) and chemiluminescent assays (CLA) (Luminescent Assays, Perspectives in Endocrinology and Clinical Chemistry, Vol. 1, Mario Serio and Mario Pazzagli, Raven Press, 1982), (Bioluminescence and Chemiluminescense, Basic Chemistry and Analytical Applications, Marlene, A. DeLuca and William D. McElroy, Academic Press, 1981), (Journal of Bioluminescence, Vol. 4, M. Pazzagli, et al., Proceedings of the Vth International Symposium on Bioluminescence and Chemiluminescence, Wiley, 1989), etc. Numerous method variations and devices for performing such assays are available, are known to those familiar with the art, and can be found in the scientific and patent literature.

Immunoassays can be heterogeneous or homogeneous. Heterogeneous immunoassays have been applied to both small and large molecular weight analytes and involve separation of bound materials (to be detected or determined) from free materials (which can interfere with that determination). Heterogeneous immunoassays can comprise an antibody or an antigen immobilized on a solid surface such as plastic microtiter plates, beads, tubes, or the like or on membrane sheets, chips and pieces of glass, nylon, cellulose or the like (Immobilized Enzymes, Antigens, Antibodies, and Peptides, Howard H. Weetall, Marcel Dekker, Inc., 1975). In heterogeneous immunoassays, antigen-antibody complexes bound to the solid phase are separated from unreacted and non-specific analyte in solution, generally by centrifugation, filtration, precipitation, magnetic separation or aspiration of fluids from solid phases, followed by repeated washing of the solid phase bound antigen-antibody complex. Of particular interest are immunometric “sandwich” assays (Immunochemistry of Solid-Phase Immunoassay, John E. Butler, CRC Press, 1991) which first require binding of an immobilized antigen or antibody with the target analyte (e.g., the target antigen or target antibody) from the biological sample. Separation of the immobilized complex and subsequent repeated washing is followed by the introduction of a secondary binding agent specific to the target analyte, said secondary binding agents usually being chemically conjugated with radioisotopes, enzyme, fluorescent or chemiluminescent labels described earlier. Secondary binding agents are typically immunoglobulin antibodies, antibody fragments, monoclonal antibodies or recombinant antibodies. The analyte is “sandwiched” between the first immobilized antigen or antibody and the labeled secondary binding agent. Subsequent separation and washing are used to remove unbound labeled secondary binding agents. Direct measurement of the labeled, immobilized bound complex or indirect measurement with the use of substrates is then undertaken. In contrast to heterogeneous immunoassays, homogenous immunoassays are, in general, liquid phase procedures where antigens or antibodies that bind to a target analyte are not immobilized on solid materials.

Immunoassays can be useful diagnostic tools in determining whether or not a particular individual has been exposed to an infectious disease. For example, individuals exposed to a pathogenic agent (e.g., a virus) that causes a disease can develop antibodies to various epitopes of the pathogenic agent, and later detection of these antibodies in samples from the individual can be used to confirm that the individual has been exposed to the agent. Thus, for example, antibodies specific to a particular pathogen present in a biological sample from a patient (e.g., a blood, serum or plasma sample) can be an analyte of interest (or “target analyte” or “target antibody”) in an immunoassay. Thus, in the context of a heterogenous immunoassay, an antigenic protein or peptide associated with the pathogenic agent, i.e., a protein or peptide comprising one or more epitopes, can be immobilized on a solid surface and exposed to a sample potentially comprising anti-pathogen antibodies. However, in the case of detection for anti-SARS-CoV-2 antibodies (i.e., “COVID-19 antibodies”), the presence of common epitopes among the coronavirus family of viruses can make accurate confirmation of prior SARS-CoV-2 infection difficult.

Generally, coronaviruses are enveloped, positive-sense single-stranded RNA viruses. They have the largest genomes (26-32 kb) among known RNA viruses, and are phylogenetically divided into four genera (alpha (a), beta (13), gamma (γ), and delta (6)), with betacoronaviruses further subdivided into four lineages (A, B, C, D). Coronaviruses infect a wide range of avian and mammalian species, including humans. Of the six known human coronaviruses, four of them (HCoV-OC43, HCoV-229E, HCoV-HKU1 and HCoV-NL63) circulate annually in humans and generally cause mild respiratory diseases, although severity can be greater in infants, elderly, and the immunocompromised.

Thus, although SARS-CoV-2 is novel, other members of the Coronavirdae family are widespread pathogens that are the second most common cause of the “common cold” as well as a wide variety of upper and lower respiratory symptomatologies. See Su et al. 2016. Because coronaviruses can have antigenic similarities, the ubiquitous nature of Coronavirdae raises the problem that antibodies against common coronaviruses can cross react with more pathogenic variants. Indeed, globally, greater than 90% of humans, and up to 99% of patients with respiratory pathology, have antibodies against one or more of the four common coronaviruses (OC43-CoV, HKU1-CoV, NL63-CoV and 229E-CoV). See Gorse et al., 2010. Thus, cross reactivity of anti-SARS-CoV-2 antibodies against the four common coronaviruses can be a complication for serological testing. See Meyer et al., 2014. Particular examples are well documented. For example, in 2006 an outbreak of the common coronavirus OC43-CoV at a nursing home in British Columbia was mistaken for SARS-CoV, because antibodies to OC43-CoV cross reacted with the SARS-CoV antigens used in assays to detect antibodies against SARS-CoV. See Patrick et al., 2006.

The two most immunogenic components of coronaviruses are the Spike protein (S) and the Nucleocapsid protein (N), and these two proteins can be used as the antigen targets in serological immunoassays. In general, the S protein is more variant among the various coronaviruses, and as such is less susceptible to concerns of cross-reactivity. However, antibodies to the S protein are also typically less prevalent in patient samples, which can result in decreased sensitivity in laboratory testing. In contrast, while antibodies to N proteins are far more easily detected (high sensitivity), the problem of cross reactivity can lead to lower specificity. Thus, patients actually seronegative for the SARS-CoV-2 can be mistakenly labeled as seropositive because of antibodies to one or more of the common cold coronaviridae. See Patrick et al., 2006.

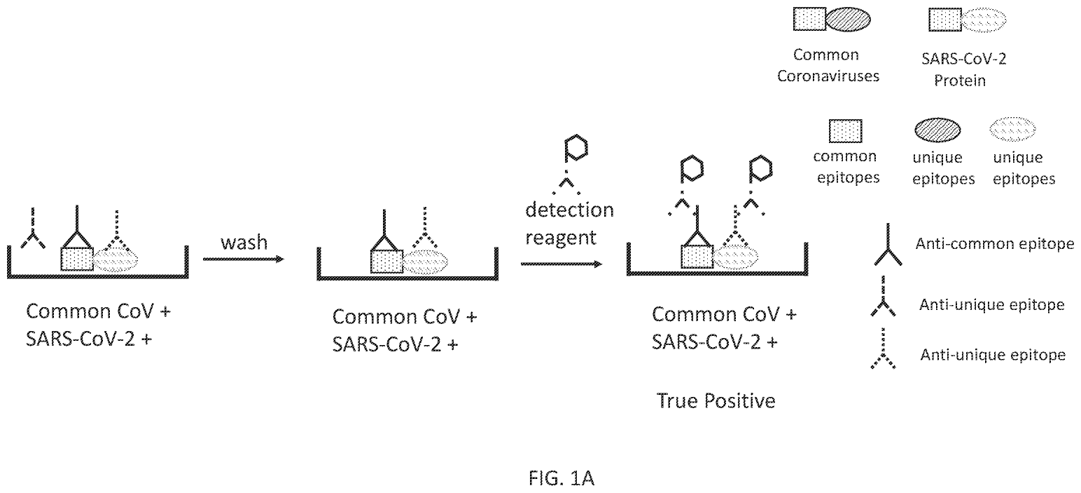

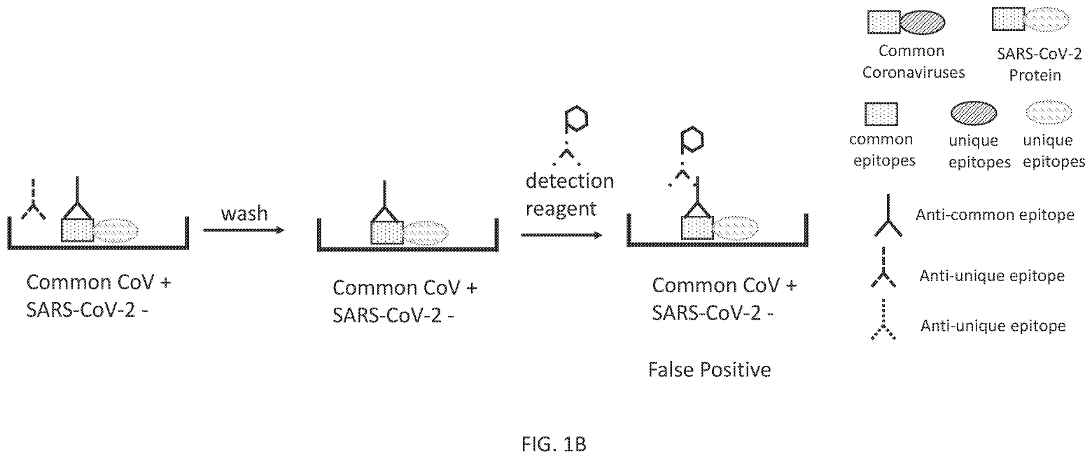

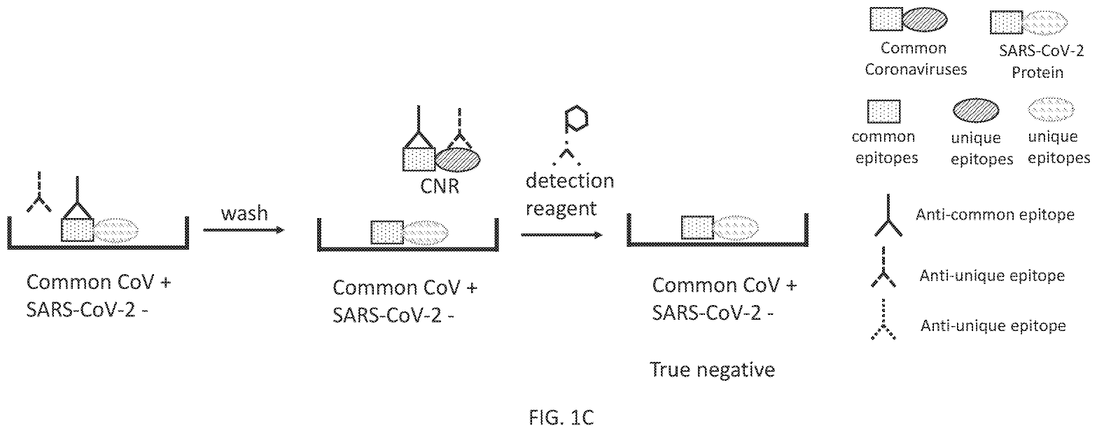

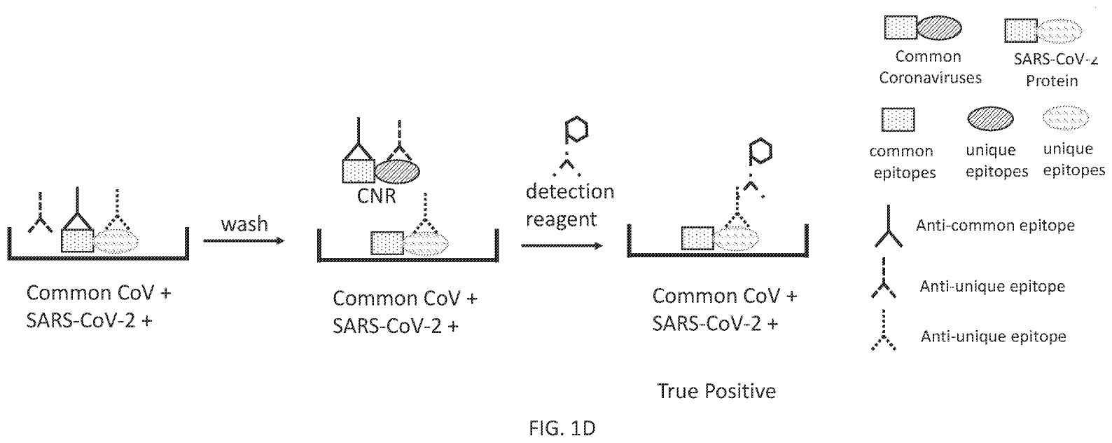

Possible serological scenarios are shown in FIGS. 1A and 1B. FIG. 1A shows a schematic diagram for an immunoassay for detecting anti-SARS-CoV-2 antibodies in a sample from a patient who has been infected with SARS-CoV-2 and has developed antibodies to SARS-CoV-2 (i.e., is SARS-CoV-2+). The patient also has antibodies to common coronaviruses (i.e., is Common CoV+), e.g., from prior infections of common coronaviruses. Thus, the patient can have anti-common epitope antibodies (antibodies that specifically bind one or more epitopes common to both common coronaviruses and to SARS-CoV-2), as well as different types of anti-unique epitope antibodies, including anti-unique epitope antibodies that specifically bind one or more epitopes unique to one or more common coronavirus and anti-unique epitope antibodies that specifically bind one or more epitopes unique to SARS-CoV-2. At the left of FIG. 1A, a sample containing the patient's antibodies (including anti-common epitope antibodies and anti-unique epitope antibodies) is added to a container (e.g., a well in a microtiter plate) containing an immobilized SARS-CoV-2 protein (e.g., a SARS-CoV-2 nucleocapsid protein or fragment thereof or a SARS-CoV-2 spike protein or fragment thereof) as a “target antigen.” The immobilized SARS-CoV-2 protein can include epitopes that are common to many coronaviruses (e.g., including common coronaviruses), as well as epitopes that are unique to SARS-CoV-2. Anti-common epitope antibodies and anti-unique epitope antibodies that specifically bind epitopes unique to SARS-CoV-2 can form complexes with the immobilized SARS-CoV-2 protein, while antibodies directed to unique epitopes of common coronaviruses do not form complexes with the immobilized SARS-CoV-2 protein and can be removed, e.g., in a washing step. See middle of FIG. 1A. Addition of a detection reagent, such as a detectable secondary antibody that binds to the Fc region of the antibodies in the patient sample (e.g., an antibody directed to human Fc regions linked to a detectable tag or label) results in further complexes being formed between the detection reagent and the previously formed antibody/SARS-CoV-2 protein complexes. In this scenario, signal provided by the detection reagent provides a “true positive” result, i.e., in that a patient who has been exposed to SARS-CoV-2 will be correctly identified as having anti-SARS-CoV-2 antibodies.

However, due to the presence of common epitopes among coronaviruses, there is a chance of false positive results, as shown in FIG. 1B. As shown at the left of FIG. 1B, a sample containing antibodies from a patient who has developed antibodies to common coronaviruses (Common CoV+) but who has not been exposed to SARS-CoV-2 (SARS-CoV-2-) can contain both anti-common epitope antibodies and anti-unique epitope antibodies that specifically bind to unique epitopes of common coronaviruses. Incubation of the sample with immobilized SARS-CoV-2 protein results in a complex being formed between the common epitopes in the SARS-CoV-2 protein and the anti-common epitope antibodies. See FIG. 1B, middle. These antibodies will not be removed during a washing step. Addition of the detection reagent will result in a detectable complex being formed, mistakenly indicating the presence of anti-SARS-CoV-2 antibodies and identifying the subject as having been exposed to SARS-CoV-2.

### III. Representative Compositions and Methods

The presently disclosed subject matter provides methods of detecting antibodies for SARS-CoV-2 (i.e., anti-SARS-CoV-2 antibodies, which can also be referred to herein as “COVID-19 antibodies”, antibodies that specifically bind to SARS-CoV-2), as well as to related reagents, including mutant SARS-CoV-2 proteins. The methods and/or reagents can provide improved specificity for the detection of anti-SARS-CoV-2 antibodies.

### III.A. Cross Reactivity Neutralizing Regent (CNR)

In some embodiments, the presently disclosed subject matter relates to the use of proteins (or protein fragments) from one or more of the common coronaviruses (OC43-CoV, HKU1-CoV, NL63-CoV, and 229E-CoV) in an assay for detecting antibodies to the SARS-CoV-2 virus. In some embodiments, the proteins are N and/or S proteins from common coronaviruses. These common coronavirus protein reagents can remove antibodies from samples (e.g., patient samples) that can otherwise cross-react with antigens for SARS-CoV-2.

In some embodiments, the presently disclosed subject matter relates to the recombinant expression of common coronavirus proteins (e.g., the N and/or S proteins) from one or more (or each) of the known common coronaviruses (OC43-CoV, HKU1-CoV, NL63-CoV, and 229E-CoV). These can be expressed in bacteria or eukaryotic cells (e.g., HEK 293), the latter used to maintain human post-translational modifications, such as glycosylation. In either case, the proteins can be expressed as fusions with epitope tags (e.g., a FLAG tag, a His tag, a glutathione-S-transferase (GST) tag, etc.) in order to allow rapid purification. In some embodiments, tags can themselves be cleavable by sequence specific proteases to allow ready removal of the tag from the protein (e.g., the N or S protein) to which it is fused. The tags can be fused to either the N or C end of the common coronavirus protein (e.g. the N or S protein). As described further hereinbelow, recombinant proteins to common coronaviruses can also be obtained from commercial sources.

Prior to or during testing in an immunoassay for detecting anti-SARS-CoV-2 antibodies, a sample from a human or other animal subject that contains circulating antibodies (e.g., a blood or serum sample) can be incubated with one or more recombinant common coronavirus protein (e.g., a common coronavirus N or S protein) either individually or as a cocktail. These recombinant common coronavirus proteins and their admixtures are henceforth referred to herein as “Cross-reactivity Neutralizing Reagents” (CNRs)

For instance, in some embodiments, in an assay where a SARS-CoV-2 protein antigen is immobilized on a solid support, when a sample from a human or other animal subject is mixed with one or more CNR, any antibodies present in the sample that are reactive with the CNR (e.g., recombinant N or S common coronavirus proteins) can be bound in the fluid phase prior to, during, or after incubation of the sample with an immobilized SARS-CoV-2 antigen and are prevented from forming a complex with the bound SARS-CoV-2 antigen or removed from such a complex. Any antibodies in the sample that are specific for SARS-CoV-2 antigens can escape binding to the recombinant CNR and can be free to bind or remain bound to the SARS-CoV-2 antigen bound to the solid support. Antibodies that cross-react with the CNR can be removed during a conventional immunoassay wash step, whereas antibodies specific for SARS-CoV-2 can remain bound to the immobilized antigen. The specific anti-SARS-CoV-2 antibodies can then be detected by a standard detection reagent (e.g., a secondary antibody comprising a detectable label).

Because over 90% of people have antibodies to common coronavirus variants, for most people, antibodies to common coronaviruses are always present, whether or not there are antibodies to SARS-CoV-2. Accordingly, in some aspects, use of a CNR can result in two possible scenarios. In the first scenario, cross-reactive antibodies in a subject sample from one or more previous cold virus infections (e.g., with OC43-CoV, HKU1-CoV, NL63-CoV, and/or 229E-CoV) can be removed. This can produce the desired effect of increasing the specificity of the immunoassay (e.g., reducing false positives) by removing antibodies that were not the result of a SARS-CoV-2 infection, but would otherwise have been mistaken for anti-SARS-CoV-2 antibodies. In the second scenario, antibodies in the sample that are the product of a SARS-CoV-2 infection but that are cross-reactive with common coronaviruses (e.g., OC43-CoV, HKU1-CoV, NL63-CoV, and 229E-CoV) can be removed, resulting in decreased sensitivity of the immunoassay (i.e., reducing the number of true positives) by removing some of the authentic anti-SARS-CoV-2 antibodies. However, it is predicted that the loss of sensitivity will be modest, and the increased specificity can improve the diagnostic utility of serologic assays that employ the CNR incubation (e.g., preincubation) step.

The use of CNRs is further demonstrated in FIGS. 1C and 1D. For example, in FIG. 1C, the patient has antibodies resulting from exposure to common coronaviruses (i.e., anti-common epitope antibodies and anti-unique epitope antibodies that specifically bind to unique epitopes of one or more common coronavirus). See FIG. 1C, left. When contacted to an immobilized SARS-CoV-2 protein (e.g., a SARS-CoV-2 N protein or a SARS-CoV-2 S protein), a complex is formed between the anti-common epitope antibodies in the sample and the common epitopes present in the SARS-CoV-2 protein immobilized on the solid support. Normally, washing would only remove the anti-unique epitope antibodies. However, if a CNR (e.g., a N or S protein comprising both common coronavirus epitopes and unique epitopes associated with one or more of the common coronaviruses) is used, it can form complexes with both the anti-unique epitope antibodies and the anti-common antibodies, resulting in their removal. Thus, when contacted with the detection reagent, no additional complex is formed, resulting in no signal and a true negative result.

FIG. 1D shows the same method performed in the case of a sample from a patient that contains both common coronavirus antibodies and authentic SARS-CoV-2 antibodies (i.e., which include anti-unique epitope antibodies specific for unique epitopes of SARS-CoV-2). Again, use of the CNR removes both (i) anti-unique epitope antibodies that are specific to unique epitopes of common coronaviruses and (ii) anti-common epitope antibodies. In this case, some of the anti-common epitope antibodies can be present as the result of a SARS-CoV-2 infection experienced by the patient and thus can be considered authentic SARS-CoV-2 antibodies. However, the CNR will not form a complex with the anti-unique epitope antibodies that are specific for unique epitopes of SARS-CoV-2. These will remain complexed to the SARS-CoV-2 protein immobilized on the plate. After addition of a detection reagent, a signal will be generated. A “true positive” result will be provided, albeit with the possibility that signal strength could be decreased, thereby reducing sensitivity.

Accordingly, in some embodiments the presently disclosed subject matter provides a method of performing an immunoassay to detect a presence or absence of an antibody for SARS-CoV-2 (i.e., a COVID-19 antibody) in a sample comprising antibodies. In some embodiments, the sample is a biological sample from a subject, e.g., a mammalian or avian subject. In some embodiments, the subject is a human subject. In some embodiments, the subject is a subject suspected of having had COVID-19 or having been exposed to SARS-CoV-2. In some embodiments, the sample is a blood sample or a serum sample.

In some embodiments, the method comprises incubating the sample with a CNR under conditions sufficient to form an antibody/CNR complex between the CNR and any antibody present in the sample specific for the CNR; incubating the sample with a SARS-CoV-2 protein or fragment thereof under conditions sufficient to form an antibody/SARS-CoV-2 protein complex between the SARS-CoV-2 protein or fragment thereof and any antibody in the sample specific for the SARS-CoV-2 protein or fragment thereof; treating the sample to remove any antibody/CNR complex present in the sample, thereby forming a subtracted sample; and analyzing the subtracted sample to determine the presence or absence of any antibody/SARS-CoV-2 protein or protein complex, thereby detecting a presence or absence of antibody binding to the SARS-CoV-2 protein or fragment thereof.

The CNR can comprise, consist essentially of, or consist of one or more proteins or protein fragments from a common coronavirus. In some embodiments, the common coronavirus proteins or protein fragments are selected from the group comprising a spike protein, a spike protein fragment, a nucleocapsid protein, and a nucleocapsid protein fragment. In some embodiments, the CNR comprises a plurality of such proteins or protein fragments or a fusion protein thereof. In some embodiments, the CNR comprises, consists essentially of, or consists of one or more recombinant proteins or protein fragments.

In some embodiments, the common coronavirus is selected from OC43-CoV, HKU1-CoV, NL63-CoV, and 229E-CoV. Amino acid and nucleic acid sequences for common coronavirus nucleocapsid protein and spike proteins are known in the art. For example, exemplary amino acid sequences of nucleocapsid and spike proteins for OC43-CoV, HKU1-CoV, NL63-CoV, and 229E-CoV are summarized in Tables 3 and 4, below:

TABLE 3
Common Coronavirus Nucleocapsid Protein Sequences

| | | Amino Acid |
|---|---|---|
| Coronavirus | Nucleic Acid Sequence | Sequence |
| Human | nucleotides 29079-30425 of | GENBANK ® |
| coronavirus | GENBANK ® Accession No. | Accession No. |
| OC43 | NC_006213.1 (Human | YP_009555245.1; |
| | coronavirus OC43 strain | SEQ ID NO: 2 |
| | ATCC VR-759, complete genome); | |
| | SEQ ID NO: 1 | |
| Human | nucleotides 28320-29645 of | GENBANK ® |
| coronavirus | GENBANK ® Accession No. | Accession No. |
| HKU1 | NC_006577.2 (Human | YP_173242.1; |
| | coronavirus HKU1, complete genome); | SEQ ID NO: 4 |
| | SEQ ID NO: 3 | |
| Human | nucleotides 26133-27266 of | GENBANK ® |
| coronavirus | GENBANK ® Accession No. | Accession No. |
| NL63 | NC_005831.2 (Human | YP_003771.1; |
| | Coronavirus NL63, complete genome); | SEQ ID NO: 6 |
| | SEQ ID NO: 5 | |
| Human | nucleotides 25686-26855 of | GENBANK ® |
| coronavirus | GENBANK ® Accession No. | Accession No. |
| 299E | AF304460.1 (Human | AGW80953.1; |
| | Coronavirus 229E, complete genome); | SEQ ID NO: 8 |
| | SEQ ID NO: 7 | |
| Human | nucleotides 25686-26855 | GENBANK ® |
| coronavirus | of GENBANK ® Accession No. | Accession No. |
| 299E | NC_002645.1 (Human | NP_073556.1; |
| | Coronavirus 229E, complete genome); | SEQ ID NO: 10 |
| | SEQ ID NO: 9 | |

TABLE 4
Common Coronavirus Spike Protein Sequences

| | | Amino Acid |
|---|---|---|
| Coronavirus | Nucleic Acid Sequence | Sequence |
| Human | nucleotides 23643-27704 of | GENBANK ® |
| coronavirus | GENBANK ® Accession No. | Accession No. |
| OC43 | NC_006213.1 (Human coronavirus | YP_009555241.1; |
| | OC43 strain ATCC VR-759, | SEQ ID NO: 14 |
| | complete genome); SEQ ID NO: 13 | |
| Human | nucleotides 22942-27012 of | GENBANK ® |
| coronavirus | GENBANK ® Accession No. | Accession No. |
| HKU1 | NC_006577.2 (Human coronavirus | YP_173238.1; |
| | HKU1, complete genome); | SEQ ID NO: 16 |
| | SEQ ID NO: 15 | |
| Human | nucleotides 20472-24542 of | GENBANK ® |
| coronavirus | GENBANK ® Accession No. | Accession No. |
| NL63 | NC_005831.2 (Human Coronavirus | YP_003767.1; |
| | NL63, complete genome); | SEQ ID NO: 18 |
| | SEQ ID NO: 17 | |
| Human | nucleotides 20570-24091 of | GENBANK ® |
| coronavirus | GENBANK ® Accession No. | Accession No. |
| 299E | NC_002645.1 (Human Coronavirus | NP_073551.1; |
| | 229E, complete genome); | SEQ ID NO: 20 |
| | SEQ ID NO: 19 | |

More particularly, the exemplary amino acid sequences for the nucleocapsid proteins and spike proteins of OC43-CoV, HKU1-CoV, NL63-CoV, and 229E-CoV are as follows:

OC43-CoV nucleocapsid protein (SEQ ID NO: 2):
MSFTPGKQSSSRASSGNRSGNGILKWADQSDQFRNVQTRGRRAQPKQTA
TSQQPSGGNVVPYYSWFSGITQFQKGKEFEFVEGQGVPIAPGVPATEAK
GYWYRHNRRSFKTADGNQRQLLPRWYFYYLGTGPHAKDQYGTDIDGVYW
VASNQADVNTPADIVDRDPSSDEAIPTRFPPGTVLPQGYYIEGSGRSAP
NSRSTSRTSSRASSAGSRSRANSGNRTPTSGVTPDMADQIASLVLAKLG
KDATKPQQVTKHTAKEVRQKILNKPRQKRSPNKQCTVQQCFGKRGPNQN
FGGGEMLKLGTSDPQFPILAELAPTAGAFFFGSRLELAKVQNLSGNPDE
PQKDVYELRYNGAIRFDSTLSGFETIMKVLNENLNAYQQQDGMMNMSPK
PQRQRGHKNGQGENDNISVAVPKSRVQQNKSRELTAEDISLLKKMDEPY
TEDTSEI
HKU1-CoV nucleocapsid protein (SEQ ID NO: 4):
MSFTPGKQSSSRASSGNRSGNGILKWADQSDQFRNVQTRGRRAQPKQTA
TSQQPSGGNVVPYYSWFSGITQFQKGKEFEFVEGQGVPIAPGVPATEAK
GYWYRHNRRSFKTADGNQRQLLPRWYFYYLGTGPHAKDQYGTDIDGVYW
VASNQADVNTPADIVDRDPSSDEAIPTRFPPGTVLPQGYYIEGSGRSAP
NSRSTSRTSSRASSAGSRSRANSGNRTPTSGVTPDMADQIASLVLAKLG
KDATKPQQVTKHTAKEVRQKILNKPRQKRSPNKQCTVQQCFGKRGPNQN
FGGGEMLKLGTSDPQFPILAELAPTAGAFFFGSRLELAKVQNLSGNPDE
PQKDVYELRYNGAIRFDSTLSGFETIMKVLNENLNAYQQQDGMMNMSPK
PQRQRGHKNGQGENDNISVAVPKSRVQQNKSRELTAEDISLLKKMDEPY
TEDTSEI
NL63-CoV nucleocapsid protein (SEQ ID NO: 6):
MASVNWADDRAARKKFPPPSFYMPLLVSSDKAPYRVIPRNLVPIGKGNK
DEQIGYWNVQERWRMRRGQRVDLPPKVHFYYLGTGPHKDLKFRQRSDGV
VWVAKEGAKTVNTSLGNRKRNQKPLEPKFSIALPPELSVVEFEDRSNNS
SRASSRSSTRNNSRDSSRSTSRQQSRTRSDSNQSSSDLVAAVTLALKNL
GFDNQSKSPSSSGTSTPKKPNKPLSQPRADKPSQLKKPRWKRVPTREEN
VIQCFGPRDFNHNMGDSDLVQNGVDAKGFPQLAELIPNQAALFFDSEVS
TDEVGDNVQITYTYKMLVAKDNKNLPKFIEQISAFTKPSSIKEMQSQSS
HVAQNTVLNASIPESKPLADDDSAIIEIVNEVLH
229E-CoV nucleocapsid protein (SEQ ID NO: 8):
MATVKWADASEPQRGRQGRIPYSLYSPLLVDSEQPWKVIPRNLVPINKK
DKNKLIGYWNVQKRFRTRKGKRVDLSPKLHFYYLGTGPHKDAKFRERVE
GVVWVAVDGAKTEPTGYGVRRKNSEPEIPHFNQKLPNGVTVVEEPDSRA
PSRSQSRSQSRGRGESKPQSRNPSSDRNHNSQDDIMKAVAAALKSLGFD
KPQEKDKKSAKTGTPKPSRNQSPASSQTSAKSLARSQSSETKEQKHEMQ
KPRWKRQPNDDVTSNVTQCFGPRDLDHNFGSAGVVANGVKAKGYPQFAE
LVPSTAAMLFDSHIVSKESGNTVVLTFTTRVTVSKDHPHLGKFLEELNA
FTREMQQHPLLNPSALEFNPSQTSPATAEPVRDEVSIETDIIDEVN
229E-CoV nucleocapsid protein (SEQ ID NO: 10):
MATVKWADASEPQRGRQGRIPYSLYSPLLVDSEQPWKVIPRNLVPINKK
DKNKLIGYWNVQKRFRTRKGKRVDLSPKLHFYYLGTGPHKDAKFRERVE
GVVWVAVDGAKTEPTGYGVRRKNSEPEIPHFNQKLPNGVTVVEEPDSRA
PSRSQSRSQSRGRGESKPQSRNPSSDRNHNSQDDIMKAVAAALKSLGFD
KPQEKDKKSAKTGTPKPSRNQSPASSQTSAKSLARSQSSETKEQKHEMQ
KPRWKRQPNDDVTSNVTQCFGPRDLDHNFGSAGVVANGVKAKGYPQFAE
LVPSTAAMLFDSHIVSKESGNTVVLTFTTRVTVPKDHPHLGKFLEELNA
FTREMQQHPLLNPSALEFNPSQTSPATAEPVRDEVSIETDIIDEVN
OC43-CoV spike protein (SEQ ID NO: 14):
MFLILLISLPTAFAVIGDLKCTSDNINDKDTGPPPISTDTVDVTNGLGT
YYVLDRVYLNTTLFLNGYYPTSGSTYRNMALKGSVLLSRLWFKPPFLSD
FINGIFAKVKNTKVIKDRVMYSEFPAITIGSTFVNTSYSVVVQPRTINS
TQDGDNKLQGLLEVSVCQYNMCEYPQTICHPNLGNHRKELWHLDTGVVS
CLYKRNFTYDVNADYLYFHFYQEGGTFYAYFTDTGVVTKFLFNVYLGMA
LSHYYVMPLTCNSKLTLEYWVTPLTSRQYLLAFNQDGIIFNAVDCMSDF
MSEIKCKTQSIAPPTGVYELNGYTVQPIADVYRRKPNLPNCNIEAWLND
KSVPSPLNWERKTFSNCNFNMSSLMSFIQADSFTCNNIDAAKIYGMCFS
SITIDKFAIPNGRKVDLQLGNLGYLQSFNYRIDTTATSCQLYYNLPAAN
VSVSRFNPSTWNKRFGFIEDSVFKPRPAGVLTNHDVVYAQHCFKAPKNF
CPCKLNGSCVGSGPGKNNGIGTCPAGTNYLTCDNLCTPDPITFTGTYKC
PQTKSLVGIGEHCSGLAVKSDYCGGNSCTCRPQAFLGWSADSCLQGDKC
NIFANFILHDVNSGLTCSTDLQKANTDIILGVCVNYDLYGILGQGIFVE
VNATYYNSWQNLLYDSNGNLYGFRDYITNRTFMIRSCYSGRVSAAFHAN
SSEPALLFRNIKCNYVFNNSLTRQLQPINYFDSYLGCVVNAYNSTAISV
QTCDLTVGSGYCVDYSKNRRSRGAITTGYRFTNFEPFTVNSVNDSLEPV
GGLYEIQIPSEFTIGNMVEFIQTSSPKVTIDCAAFVCGDYAACKSQLVE
YGSFCDNINAILTEVNELLDTTQLQVANSLMNGVTLSTKLKDGVNFNVD
DINFSPVLGCLGSECSKASSRSAIEDLLFDKVKLSDVGFVEAYNNCTGG
AEIRDLICVQSYKGIKVLPPLLSENQISGYTLAATSASLFPPWTAAAGV
PFYLNVQYRINGLGVTMDVLSQNQKLIANAFNNALYAIQEGFDATNSAL
VKIQAVVNANAEALNNLLQQLSNRFGAISASLQEILSRLDALEAEAQID
RLINGRLTALNAYVSQQLSDSTLVKFSAAQAMEKVNECVKSQSSRINFC
GNGNHIISLVQNAPYGLYFIHFSYVPTKYVTARVSPGLCIAGDRGIAPK
SGYFVNVNNTWMYTGSGYYYPEPITENNVVVMSTCAVNYTKAPYVMLNT
SIPNLPDFKEELDQWFKNQTSVAPDLSLDYINVTFLDLQVEMNRLQEAI
KVLNQSYINLKDIGTYEYYVKWPWYVWLLICLAGVAMLVLLFFICCCTG
CGTSCFKKCGGCCDDYTGYQELVIKTSHDD
HKU1-CoV spike protein (SEQ ID NO. 16):
MLLIIFILPTTLAVIGDFNCTNFAINDLNTTVPRISEYVVDVSYGLGTY
YILDRVYLNTTILFTGYFPKSGANFRDLSLKGTTYLSTLWYQKPFLSDF
NNGIFSRVKNTKLYVNKTLYSEFSTIVIGSVFINNSYTIVVQPHNGVLE
ITACQYTMCEYPHTICKSKGSSRNESWHFDKSEPLCLFKKNFTYNVSTD
WLYFHFYQERGTFYAYYADSGMPTTFLFSLYLGTLLSHYYVLPLTCNAI
SSNTDNETLQYWVTPLSKRQYLLKFDNRGVITNAVDCSSSFFSEIQCKT
KSLLPNTGVYDLSGFTVKPVATVHRRIPDLPDCDIDKWLNNFNVPSPLN
WERKIFSNCNFNLSTLLRLVHTDSFSCNNFDESKIYGSCFKSIVLDKFA
IPNSRRSDLQLGSSGFLQSSNYKIDTTSSSCQLYYSLPAINVTINNYNP
SSWNRRYGFNNFNLSSHSVVYSRYCFSVNNTFCPCAKPSFASSCKSHKP
PSASCPIGTNYRSCESTTVLDHTDWCRCSCLPDPITAYDPRSCSQKKSL
VGVGEHCAGFGVDEEKCGVLDGSYNVSCLCSTDAFLGWSYDTCVSNNRC
NIFSNFILNGINSGTTCSNDLLQPNTEVFTDVCVDYDLYGITGQGIFKE
VSAVYYNSWQNLLYDSNGNIIGFKDFVTNKTYNIFPCYAGRVSAAFHQN
ASSLALLYRNLKCSYVLNNISLTTQPYFDSYLGCVFNADNLTDYSVSSC
ALRMGSGFCVDYNSPSSSSSRRKRRSISASYRFVTFEPFNVSFVNDSIE
SVGGLYEIKIPTNFTIVGQEEFIQTNSPKVTIDCSLFVCSNYAACHDLL
SEYGTFCDNINSILDEVNGLLDTTQLHVADTLMQGVTLSSNLNTNLHFD
VDNINFKSLVGCLGPHCGSSSRSFFEDLLFDKVKLSDVGFVEAYNNCTG
GSEIRDLLCVQSFNGIKVLPPILSESQISGYTTAATVAAMFPPWSAAAG
IPFSLNVQYRINGLGVTMDVLNKNQKLIATAFNNALLSIQNGFSATNSA
LAKIQSVVNSNAQALNSLLQQLFNKFGAISSSLQEILSRLDALEAQVQI
DRLINGRLTALNAYVSQQLSDISLVKFGAALAMEKVNECVKSQSPRINF
CGNGNHILSLVQNAPYGLLFMHFSYKPISFKTVLVSPGLCISGDVGIAP
KQGYFIKHNDHWMFTGSSYYYPEPISDKNVVFMNTCSVNFTKAPLVYLN
HSVPKLSDFESELSHWFKNQTSIAPNLTLNLHTINATFLDLYYEMNLIQ
ESIKSLNNSYINLKDIGTYEMYVKWPWYVWLLISFSFIIFLVLLFFICC
CTGCGSACFSKCHNCCDEYGGHHDFVIKTSHDD
NL63-CoV spike protein (SEQ ID NO. 18):
MKLFLILLVLPLASCFFTCNSNANLSMLQLGVPDNSSTIVTGLLPTHWF
CANQSTSVYSANGFFYIDVGNHRSAFALHTGYYDANQYYIYVTNEIGLN
ASVTLKICKFSRNTTFDFLSNASSSFDCIVNLLFTEQLGAPLGITISGE
TVRLHLYNVTRTFYVPAAYKLTKLSVKCYFNYSCVFSVVNATVTVNVTT
HNGRVVNYTVCDDCNGYTDNIFSVQQDGRIPNGFPFNNWFLLTNGSTLV
DGVSRLYQPLRLTCLWPVPGLKSSTGFVYFNATGSDVNCNGYQHNSVVD
VMRYNLNFSANSLDNLKSGVIVFKTLQYDVLFYCSNSSSGVLDTTIPFG
PSSQPYYCFINSTINTTHVSTFVGILPPTVREIVVARTGQFYINGFKYF
DLGFIEAVNFNVTTASATDFWTVAFATFVDVLVNVSATNIQNLLYCDSP
FEKLQCEHLQFGLQDGFYSANFLDDNVLPETYVALPIYYQHTDINFTAT
ASFGGSCYVCKPHQVNISLNGNTSVCVRTSHFSIRYIYNRVKSGSPGDS
SWHIYLKSGTCPFSFSKLNNFQKFKTICFSTVEVPGSCNFPLEATWHYT
SYTIVGALYVTWSEGNSITGVPYPVSGIREFSNLVLNNCTKYNIYDYVG
TGIIRSSNQSLAGGITYVSNSGNLLGFKNVSTGNIFIVTPCNQPDQVAV
YQQSIIGAMTAVNESRYGLQNLLQLPNFYYVSNGGNNCTTAVMTYSNFG
ICADGSLIPVRPRNSSDNGISAIITANLSIPSNWTTSVQVEYLQITSTP
IVVDCATYVCNGNPRCKNLLKQYTSACKTIEDALRLSAHLETNDVSSML
TFDSNAFSLANVTSFGDYNLSSVLPQRNIRSSRIAGRSALEDLLFSKVV
TSGLGTVDVDYKSCTKGLSIADLACAQYYNGIMVLPGVADAERMAMYTG
SLIGGMVLGGLTSAAAIPFSLALQARLNYVALQTDVLQENQKILAASFN
KAINNIVASFSSVNDAITQTAEAIHTVTIALNKIQDVVNQQGSALNHLT
SQLRHNFQAISNSIQAIYDRLDSIQADQQVDRLITGRLAALNAFVSQVL
NKYTEVRGSRRLAQQKINECVKSQSNRYGFCGNGTHIFSIVNSAPDGLL
FLHTVLLPTDYKNVKAWSGICVDGIYGYVLRQPNLVLYSDNGVFRVTSR
VMFQPRLPVLSDFVQIYNCNVTFVNISRVELHTVIPDYVDVNKTLQEFA
QNLPKYVKPNFDLTPFNLTYLNLSSELKQLEAKTASLFQTTVELQGLID
QINSTYVDLKLLNRFENYIKWPWWVWLIISVVFVVLLSLLVFCCLSTGC
CGCCNCLTSSMRGCCDCGSTKLPYYEFEKVHVQ
229E-CoV spike protein (SEQ ID NO: 20):
MFVLLVAYALLHIAGCQTTNGLNTSYSVCNGCVGYSENVFAVESGGYIP
SDFAFNNWFLLTNTSSVVDGVVRSFQPLLLNCLWSVSGLRFTTGFVYFN
GTGRGDCKGFSSDVLSDVIRYNLNFEENLRRGTILFKTSYGVVVFYCTN
NTLVSGDAHIPFGTVLGNFYCFVNTTIGNETTSAFVGALPKTVREFVIS
RTGHFYINGYRYFTLGNVEAVNFNVTTAETTDFCTVALASYADVLVNVS
QTSIANIIYCNSVINRLRCDQLSFDVPDGFYSTSPIQSVELPVSIVSLP
VYHKHTFIVLYVDFKPQSGGGKCFNCYPAGVNITLANFNETKGPLCVDT
SHFTTKYVAVYANVGRWSASINTGNCPFSFGKVNNFVKFGSVCFSLKDI
PGGCAMPIVANWAYSKYYTIGSLYVSWSDGDGITGVPQPVEGVSSFMNV
TLDKCTKYNIYDVSGVGVIRVSNDTFLNGITYTSTSGNLLGFKDVTKGT
IYSITPCNPPDQLVVYQQAVVGAMLSENFTSYGFSNVVELPKFFYASNG
TYNCTDAVLTYSSFGVCADGSIIAVQPRNVSYDSVSAIVTANLSIPSNW
TTSVQVEYLQITSTPIVVDCSTYVCNGNVRCVELLKQYTSACKTIEDAL
RNSARLESADVSEMLTFDKKAFTLANVSSFGDYNLSSVIPSLPTSGSRV
AGRSAIEDILFSKLVTSGLGTVDADYKKCTKGLSIADLACAQYYNGIMV
LPGVADAERMAMYTGSLIGGIALGGLTSAVSIPFSLAIQARLNYVALQT
DVLQENQKILAASFNKAMTNIVDAFTGVNDAITQTSQALQTVATALNKI
QDVVNQQGNSLNHLTSQLRQNFQAISSSIQAIYDRLDTIQADQQVDRLI
TGRLAALNVFVSHTLTKYTEVRASRQLAQQKVNECVKSQSKRYGFCGNG
THIFSIVNAAPEGLVFLHTVLLPTQYKDVEAWSGLCVDGTNGYVLRQPN
LALYKEGNYYRITSRIMFEPRIPTMADFVQIENCNVTFVNISRSELQTI
VPEYIDVNKTLQELSYKLPNYTVPDLVVEQYNQTILNLTSEISTLENKS
AELNYTVQKLQTLIDNINSTLVDLKWLNRVETYIKWPWWVWLCISVVLI
FVVSMLLLCCCSTGCCGFFSCFASSIRGCCESTKLPYYDVEKIHIQ

In some embodiments, the CNR comprises, consists essentially of, or consists of an amino acid sequence selected from the amino acid sequence of one of the group comprising SEQ ID NOS. 2, 4, 6, 8, 10, 14, 16, 18, and 20; or a fragment thereof. In some embodiments, the CNR comprises, consists essentially of, or consists of an amino acid sequence having at least about 70%, about 75%, about 80%, about 85%, or about 90% homology to the amino acid sequence of one of the group comprising SEQ ID NOS: 2, 4, 6, 8, 10, 14, 16, 18, and 20, or a fragment thereof. In some embodiments, the CNR comprises, consists essentially of, or consists of an amino acid sequence having at least about 95% homology (e.g., at least about 95%, about 96%, about 97%, about 98%, or about 99% homology) to the amino acid sequence of one of SEQ ID NOS: 2, 4, 6, 8, 10, 14, 16, 18, and 20, or a fragment thereof.

In some embodiments, the CNR comprises, consists essentially of, or consists of one or more nucleocapsid proteins or protein fragments. In some embodiments, the nucleocapsid protein or protein fragment is the nucleocapsid protein or fragment of a common coronavirus (e.g., OC43-CoV, HKU1-CoV, NL63-CoV, or 229E-CoV). In some embodiments, the CNR comprises, consists essentially of, or consists of an amino acid sequence selected from the amino acid sequence of SEQ ID NO: 2, SEQ ID NO: 4, SEQ ID NO: 6, SEQ ID NO: 8 or SEQ ID NO: 10, or a fragment thereof or a sequence having at least about 70%, about 75%, about 80%, about 85%, or about 90% homology thereto. In some embodiments, the CNR comprises, consists essentially of, or consists of an amino acid sequence having at least about 95% homology (e.g., about 95%, about 96%, about 97%, about 98%, or about 99% homology) to an amino acid sequence selected from the amino acid sequence selected from SEQ ID NO: 2, SEQ ID NO: 4, SEQ ID NO: 6, SEQ ID NO: 8 or SEQ ID NO: 10, or a fragment thereof.

The CNRs of the common coronaviruses can be prepared via recombinant methods known in the art or purchased from commercial sources. Spike proteins of the common coronaviruses can be purchased, for example, from BioServ UK Ltd. (Sheffield, United Kingdom), while nucleocapsid proteins are available from Native Antigen Company (Kidlington, United Kingdom; catalog numbers: REC31758 (229E-CoV); REC31856 (HKU1-CoV), REC31759 (NL63-CoV); and REC31857 (OC43-CoV)).

In some embodiments, the SARS-CoV-2 protein or fragment thereof is selected from the group comprising a SARS-COV-2 spike protein, a SARS-CoV-2 spike protein fragment; a SARS-CoV-2 nucleocapsid protein, a SARS-CoV-2 nucleocapsid protein fragment, and combinations thereof. The SARS-CoV-2 protein or fragment thereof can also be referred to herein as the “target antigen” or as the “SARS-CoV-2 antigen”. In some embodiments, the SARS-CoV-2 antigen is a recombinant protein. Typically, the target antigen and the CNR can comprise corresponding proteins or protein fragments. For instance, if the CNR comprises a common coronavirus nucleocapsid protein or fragment thereof, the target antigen can comprise a SARS-CoV-2 nucleocapsid protein or fragment thereof.

Suitable SARS-CoV-2 antigens can be prepared via recombinant methods known in the art or can be purchased from commercial sources. Table 5, below, summarizes the sequence information of exemplary SARS-CoV-2 antigens for use according to the presently disclosed subject matter.

TABLE 5
Exemplary SARS-CoV-2 Antigens

| SARS-CoV-2 | | Amino Acid |
|---|---|---|
| protein | Nucleic Acid Sequence | Sequence |
| SARS-CoV-2 | nucleotides 28274-29533 of | GENBANK ® |
| Nucleocapsid | GENBANK ® Accession No. | Accession No. |
| protein; isolate | NC_045512.2 (Severe acute | YP_009724397.2; |
| Wuhan-Hu-1 | respiratory syndrome | SEQ ID NO: 12 |
| | coronavirus 2 isolate Wuhan- | |
| | Hu-1, complete genome); | |
| | SEQ ID NO: 11 | |
| Spike protein, | nucleotides 21563-25384 of | GENBANK ® |
| isolate | GENBANK ® Accession No. | Accession No. |
| Wuhan-Hu-1 | NC_045512.2 (Severe acute | YP_009724390.1; |
| | respiratory syndrome | SEQ ID NO: 22 |
| | coronavirus 2 isolate Wuhan- | |
| | Hu-1, complete genome; | |
| | SEQ ID NO: 21 | |

More particularly, SEQ ID NO: 12 (SARS-CoV-2 nucleocapsid protein) is:

MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADL
DDFSKQLQQSMSSADSTQA

SEQ ID NO: 22 (SARS-CoV-2 spike protein) is:

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

Proteins or protein fragments comprising amino acid sequences from SARS-CoV-2 variants can also be used. SARS-CoV-2 spike protein variant amino acid sequences include, but are not limited to, SEQ ID NOS: 23-30, as follows:

(ARSCoV2 B.1.1.7 S Protein Variant amino acid sequence
(GENBANK ® Accession No. QTC27506.1)):
SEQ ID NO: 23
MFVFLVLLPLVSSQCVNLTTRTQLPPAYTNSFTRGVYYPDKVFRSSVLHSTQDLFLPFFSNVTWFHAISGTNGTKRFDNP
VLPFNDGVYFASTEKSNIIRGWIFGTTLDSKTQSLLIVNNATNVVIKVCEFQFCNDPFLGVYHKNNKSWMESEFRVYSSA
NNCTFEYVSQPFLMDLEGKQGNFKNLREFVFKNIDGYFKIYSKHTPINLVRDLPQGFSALEPLVDLPIGINITRFQTLLA
LHRSYLTPGDSSSGWTAGAAAYYVGYLQPRTFLLKYNENGTITDAVDCALDPLSETKCTLKSFTVEKGIYQTSNFRVQPT
ESIVRFPNITNLCPFGEVFNATRFASVYAWNRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLYRLFRKSNLKPFERDISTEIYQAGSTPCNGV
EGFNCYFPLQSYGFQPTYGVGYQPYRVVVLSFELLHAPATVCGPKKSTNLVKNKCVNFNFNGLTGTGVLTESNKKFLPFQ
QFGRDIDDTTDAVRDPQTLEILDITPCSFGGVSVITPGTNTSNQVAVLYQGVNCTEVPVAIHADQLTPTWRVYSTGSNVF
QTRAGCLIGAEHVNNSYECDIPIGAGICASYQTQTNSHRRARSVASQSIIAYTMSLGAENSVAYSNNSIAIPTNFTISVT
TEILPVSMTKTSVDCTMYICGDSTECSNLLLQYGSFCTQLNRALTGIAVEQDKNTQEVFAQVKQIYKTPPIKDFGGFNFS
QILPDPSKPSKRSFIEDLLFNKVTLADAGFIKQYGDCLGDIAARDLICAQKFNGLTVLPPLLTDEMIAQYTSALLAGTIT
SGWTFGAGAALQIPFAMQMAYRFNGIGVTQNVLYENQKLIANQFNSAIGKIQDSLSSTASALGKLQDVVNQNAQALNTLV
KQLSSNFGAISSVLNDILSRLDKVEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNGTHWFVTQRNFYEPQIITTDNTFVS
GNCDVVIGIVNNTVYDPLQPELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEPVLKGVKLHYT
(SARSCoV2 B.1.1.7E484K S Protein Variant amino acid sequence):
SEQ ID NO: 24
MFVFLVLLPLVSSQCVNLTTRTQLPPAYTNSFTRGVYYPDKVFRSSVLHSTQDLFLPFFSNVTWFHAISGTNGTKRFDNP
VLPFNDGVYFASTEKSNIIRGWIFGTTLDSKTQSLLIVNNATNVVIKVCEFQFCNDPFLGVYHKNNKSWMESEFRVYSSA
NNCTFEYVSQPFLMDLEGKQGNFKNLREFVFKNIDGYFKIYSKHTPINLVRDLPQGFSALEPLVDLPIGINITRFQTLLA
LHRSYLTPGDSSSGWTAGAAAYYVGYLQPRTFLLKYNENGTITDAVDCALDPLSETKCTLKSFTVEKGIYQTSNFRVQPT
ESIVRFPNITNLCPFGEVFNATRFASVYAWNRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLYRLFRKSNLKPFERDISTEIYQAGSTPCNGV
KGFNCYFPLQSYGFQPTYGVGYQPYRVVVLSFELLHAPATVCGPKKSTNLVKNKCVNFNFNGLTGTGVLTESNKKFLPFQ
QFGRDIDDTTDAVRDPQTLEILDITPCSFGGVSVITPGTNTSNQVAVLYQGVNCTEVPVAIHADQLTPTWRVYSTGSNVF
QTRAGCLIGAEHVNNSYECDIPIGAGICASYQTQTNSHRRARSVASQSIIAYTMSLGAENSVAYSNNSIAIPTNFTISVT
TEILPVSMTKTSVDCTMYICGDSTECSNLLLQYGSFCTQLNRALTGIAVEQDKNTQEVFAQVKQIYKTPPIKDFGGFNFS
QILPDPSKPSKRSFIEDLLFNKVTLADAGFIKQYGDCLGDIAARDLICAQKFNGLTVLPPLLTDEMIAQYTSALLAGTIT
SGWTFGAGAALQIPFAMQMAYRFNGIGVTQNVLYENQKLIANQFNSAIGKIQDSLSSTASALGKLQDVVNQNAQALNTLV
KQLSSNFGAISSVLNDILSRLDKVEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNGTHWFVTQRNFYEPQIITTDNTFVS
GNCDVVIGIVNNTVYDPLQPELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEPVLKGVKLHYT
(SARSCoV2 B.1.1.7P S Protein Variant amino acid sequence):
SEQ ID NO: 25
MFVFLVLLPLVSSQCVNLTTRTQLPPAYTNSFTRGVYYPDKVFRSSVLHSTQDLFLPFFSNVTWFHAISGTNGTKRFDNP
VLPFNDGVYFASTEKSNIIRGWIFGTTLDSKTQSLLIVNNATNVVIKVCEFQFCNDPFLGVYHKNNKSWMESEFRVYSSA
NNCTFEYVSQPFLMDLEGKQGNFKNLREFVFKNIDGYFKIYSKHTPINLVRDLPQGFSALEPLVDLPIGINITRFQTLLA
LHRSYLTPGDSSSGWTAGAAAYYVGYLQPRTFLLKYNENGTITDAVDCALDPLSETKCTLKSFTVEKGIYQTSNFRVQPT
ESIVRFPNITNLCPFGEVFNATRFASVYAWNRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLYRLFRKSNLKPFERDISTEIYQAGSTPCNGV
KGFNCYFPLQPYGFQPTYGVGYQPYRVVVLSFELLHAPATVCGPKKSTNLVKNKCVNFNFNGLTGTGVLTESNKKFLPFQ
QFGRDIDDTTDAVRDPQTLEILDITPCSFGGVSVITPGTNTSNQVAVLYQGVNCTEVPVAIHADQLTPTWRVYSTGSNVF
QTRAGCLIGAEHVNNSYECDIPIGAGICASYQTQTNSHRRARSVASQSIIAYTMSLGAENSVAYSNNSIAIPTNFTISVT
TEILPVSMTKTSVDCTMYICGDSTECSNLLLQYGSFCTQLNRALTGIAVEQDKNTQEVFAQVKQIYKTPPIKDFGGFNFS
QILPDPSKPSKRSFIEDLLFNKVTLADAGFIKQYGDCLGDIAARDLICAQKFNGLTVLPPLLTDEMIAQYTSALLAGTIT
SGWTFGAGAALQIPFAMQMAYRFNGIGVTQNVLYENQKLIANQFNSAIGKIQDSLSSTASALGKLQDVVNQNAQALNTLV
KQLSSNFGAISSVLNDILSRLDKVEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNGTHWFVTQRNFYEPQIITTDNTFVS
GNCDVVIGIVNNTVYDPLQPELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEPVLKGVKLHYT
(SARSCoV2 P.1a S Protein Variant amino acid sequence):
SEQ ID NO: 26
MFVFLVLLPLVSSQCVNLTTRTQLPPAYTNSFTRGVYYPDKVFRSSVLHSTQDLFLPFFSNVTWFHAIHVSGTNGTKRFD
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNSASFSTFKCYGVSPTKLNDLCFTNVYADSF
VIRGDEVRQIAPGQTGNIADYNYKLPDDFTGCVIAWNSNNLDSKVGGNYNYLYRLFRKSNLKPFERDISTEIYQAGSTPC
NGVKGFNCYFPLQSYGFQPTYGVGYQPYRVVVLSFELLHAPATVCGPKKSTNLVKNKCVNFNFNGLTGTGVLTESNKKFL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLICAQKFNGLTVLPPLLTDEMIAQYTSALLAG
TITSGWTFGAGAALQIPFAMQMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
(SARSCoV2 P.1b S Protein Variant amino acid sequence):
SEQ ID NO: 27
MFVFLVLLPLVSSQCVNLTTRTQLPPAYTNSFTRGVYYPDKVFRSSVLHSTQDLFLPFFSNVTWFHAIHVSGTNGTKRFD
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNSASFSTFKCYGVSPTKLNDLCFTNVYADSF
VIRGDEVRQIAPGQTGTIADYNYKLPDDFTGCVIAWNSNNLDSKVGGNYNYLYRLFRKSNLKPFERDISTEIYQAGSTPC
NGVKGFNCYFPLQSYGFQPTYGVGYQPYRVVVLSFELLHAPATVCGPKKSTNLVKNKCVNFNFNGLTGTGVLTESNKKFL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLICAQKFNGLTVLPPLLTDEMIAQYTSALLAG
TITSGWTFGAGAALQIPFAMQMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
(SARSCoV2B.1.351 S Protein Variant amino acid sequence):
SEQ ID NO: 28
MFVFLVLLPLVSSQCVNLTTRTQLPPAYTNSFTRGVYYPDKVFRSSVLHSTQDLFLPFFSNVTWFHAIHVSGTNGTKRFD
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNSASFSTFKCYGVSPTKLNDLCFTNVYADSF
VIRGDEVRQIAPGQTGNIADYNYKLPDDFTGCVIAWNSNNLDSKVGGNYNYLYRLFRKSNLKPFERDISTEIYQAGSTPC
NGVKGFNCYFPLQSYGFQPTYGVGYQPYRVVVLSFELLHAPATVCGPKKSTNLVKNKCVNFNFNGLTGTGVLTESNKKFL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLICAQKFNGLTVLPPLLTDEMIAQYTSALLAG
TITSGWTFGAGAALQIPFAMQMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
(SARSCoV2 B.1.427 S Protein Variant amino acid sequence
(GENBANK ® Accession No. QQX31457.1)):
SEQ ID NO: 29
MFVFLVLLPLVSSQCVNLTTRTQLPPAYTNSFTRGVYYPDKVFRSSVLHSTQDLFLPFFSNVTWFHAIHVSGTNGTKRFD
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYRYRLFRKSNLKPFERDISTEIYQAGSTPC
NGVEGFNCYFPLQSYGFQPTNGVGYQPYRVVVLSFELLHAPATVCGPKKSTNLVKNKCVNFNFNGLTGTGVLTESNKKFL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLICAQKFNGLTVLPPLLTDEMIAQYTSALLAG
TITSGWTFGAGAALQIPFAMQMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT
(SARSCoV2 B.1.429 S Protein Variant amino acid sequence
(GENBANK ® Accession No. QPJ72086.1)):
SEQ ID NO: 30
MFVFLVLLPLVSIQCVNLTTRTQLPPAYTNSFTRGVYYPDKVFRSSVLHSTQDLFLPFFSNVTWFHAIHVSGTNGTKRFD
NPVLPFNDGVYFASTEKSNIIRGWIFGTTLDSKTQSLLIVNNATNVVIKVCEFQFCNDPFLGVYYHKNNKSCMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYRYRLFRKSNLKPFERDISTEIYQAGSTPC
NGVEGFNCYFPLQSYGFQPTNGVGYQPYRVVVLSFELLHAPATVCGPKKSTNLVKNKCVNFNFNGLTGTGVLTESNKKFL
PFQQFGRDIADTTDAVRDPQTLEILDITPCSFGGVSVITPGTNTSNQVAVLYQGVNCTEVPVAIHADQLTPTWRVYSTGS
NVFQTRAGCLIGAEHVNNSYECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLICAQKFNGLTVLPPLLTDEMIAQYTSALLAG
TITSGWTFGAGAALQIPFAMQMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT

SARS-CoV-2 antigens (e.g., comprising the nucleocapsid protein, the spike protein S1 domain, the spike protein S2 domain, the spike protien S1+S2 domain, and/or the spike protein receptor binding domain (RBD)) can be purchased, for example, from Sigma Aldrich (St. Louis, Mo., United States of America) or AcroBiosystems (Newark, Del., United States of America; e.g., catalog number SPN-052H7 for spike protein and NUN-05227 for nucleocapsid protein).

In some embodiments, the SARS-CoV-2 protein or fragment thereof comprises, consists essentially of, or consists of an amino acid sequence of one of SEQ ID NOS: 12 and 22-30, or a fragment thereof. In some embodiments, the SARS-CoV-2 protein or fragment thereof comprises, consists essentially of, or consists of an amino acid sequence having at least about 70% (e.g., at least about 70%, about 75%, about 80%, about 85%, or about 90%) homology to an amino acid sequence of one of SEQ ID NOS: 12 and 22-30, or a fragment thereof. In some embodiments, the SARS-CoV-2 protein or fragment thereof comprises, consists essentially of, or consists of an amino acid sequence having at least about 95% (e.g., about 95%, about 96%, about 97%, about 98%, or about 99%) homology to an amino acid sequence of one of SEQ ID NOS: 12 and 22-30, ora fragment thereof. In some embodiments, the SARS-CoV-2 protein or fragment thereof is a nucleocapsid protein or fragment thereof or an amino acid sequence sequence having at least about 70% (e.g., at least about 70%, about 75%, about 80%, about 85%, about 90%, or at least about 95%) homology thereto. In some embodiments, the nucleocapsid protein or fragment thereof has an amino acid sequence comprising, consisting essentially of, or consisting of SEQ ID NO: 12 or a fragment thereof or a sequence having at least about 95% homology (e.g., about 95%, about 96%, about 97%, about 98%, or about 99% homology) thereto.

In some embodiments, the CNR and/or SARS-CoV-2 antigen can include a tag, such as, but not limited to a glutathione-S-transferase (GST) tag, a His tag, a FLAG tag, a hemagglutinin (HA) tag, a cMyc tag, an ALFA tag, a V5 tag, a Spot tag, a T7 tag, an NE tag and any combination thereof. Relevant sequences for the tags include: GST nucleic acid sequence (SEQ ID NO: 52): ATGTCCCCTATACTAGGTTATTGGAAAATTAAGGGCCTTGTGCAACCCAC TCGACTTCTTTTGGAATATCTTGAAGAAAAATATGAAGAGCATTTGTATG AGCGCGATGAAGGTGATAAATGGCGAAACAAAAAGTTTGAATTGGGTTT GGAGTTTCCCAATCTTCCTTATTATATTGATGGTGATGTTAAATTAACAC AGTCTATGGCCATCATACGTTATATAGCTGACAAGCACAACATGTTGGGT GGTTGTCCAAAAGAGCGTGCAGAGATTTCAATGCTTGAAGGAGCGGTTT TGGATATTAGATACGGTGTTTCGAGAATTGCATATAGTAAAGACTTTGAA ACTCTCAAAGTTGATTTTCTTAGCAAGCTACCTGAAATGCTGAAAATGTT CGAAGATCGTTTATGTCATAAAACATATTTAAATGGTGATCATGTAACCC ATCCTGACTTCATGTTGTATGACGCTCTTGATGTTGTTTTATACATGGACC CAATGTGCCTGGATGCGTTCCCAAAATTAGTTTGTTTTAAAAAACGTATT GAAGC; GST amino acid sequence (SEQ ID NO: 53): MSPILGYWKIKGLVQPTRLLLEYLEEKYEEHLYERDEGDKWRNKKFELGLE FPNLPYYIDGDVKLTQSMAIIRYIADKHNMLGGCPKERAEISMLEGAVLDIR YGVSRIAYSKDFETLKVDFLSKLPEMLKMFEDRLCHKTYLNGDHVTHPDFM LYDALDVVLYMDPMCLDAFPKLVCFKKRIEAIPQIDKYLKS SKYIAWPLQG WQATFGGGDHPPK; His Tag nucleic acid sequence (SEQ ID NO: 54): CATCACCATCACCATCAC; His Tag amino acid sequence (SEQ ID NO: 55): HHHHHH; FLAG tag amino acid sequence (SEQ ID NO: 56): DYKDDDDK; hemagglutin (HA) tag amino acid sequence (SEQ ID NO: 57): YPYDYPDYA; cMyc tag amino acid sequence (SEQ ID NO: 58): EQKLISEEDL; ALFA tag amino acid sequence (SEQ ID NO: 59): SRLEEELRRRLTE; V5 tag amino acid sequence (SEQ ID NO: 60): GKPIPNPLLGLDST; Spot tag amino acid sequence (SEQ ID NO: 61): PDRVRAVSHWSS; T7 tag amino acid sequence (SEQ ID NO: 62): MASMTGGQQMG; and NE tag amino acid sequence (SEQ ID NO: 63): TKENPRSNQEFSYDDNES. The tags can be inserted at the N terminus of the CNR or SARS-CoV-2 antigen or the C terminus. In some embodiments, the tag can be attached via an amino acid sequence that can serve as a substrate for a protease.

In some embodiments, the SARS-CoV-2 protein or fragment thereof is immobilized on a solid support. Any suitable solid support can be used. In some embodiments, the solid support is in a form selected from sticks, beads (e.g., magnetic or polymer beads), microparticles, nanoparticles, super paramagnetic particles, a microtiter or multi-well plate, a cuvette, a test tube, plastic tubing, plastic films, a lateral flow device, a flow cell, or any surface to which a protein or peptide can be passively or covalently bound. Examples of support materials on which a protein or protein fragment can be immobilized include, but are not limited to insoluble polysaccharides such as agarose and cellulose (e.g., cellulose powder), carboxymethylcellulose, dextran, silk, filter paper; synthetic resins such as silicone resins, ion exchange resins, polyamine-methyl vinyl ether-maleic acid copolymers, amino acid copolymers, ethylene-maleic acid copolymers, polystyrene resins, polyacrylamide resins, nylon resins and polycarbonate resins; and insoluble supports made of glass. In the case of a plate-form support, for example, a multi-well or microtiter plate (e.g., a 96 multi-well plate) or a biosensor chip can be used. In some embodiments, the solid support is a microtiter plate.

The SARS-CoV-2 protein or protein fragment and the support can be bound with each other by a commonly used method such as chemical binding or physical adsorption. Chemical bonding methods include, for example, covalent bonding methods e.g. diazo methods, peptide methods (acid amide derivative methods, carboxyl chloride resin method, carbodiimide resin method, maleic anhydride derivative method, isocyanate derivative method, bromocyan activated polysaccharide method, cellulose carbonate derivative method, condensing reagent method, etc.), alkylation methods, crosslinking agent coupling methods (e.g., coupling to a support using glutaraldehyde, hexamethylene isocyanate or the like as the crosslinking agent), Ugi reaction coupling methods, etc. In some embodiments, the protein or protein fragment can be immobilized by a technique selected from absorption or covalent binding with a crosslinking agent, optionally after chemical activation of the support or protein. In some embodiments, the protein or protein fragment can be immobilized on the support by immobilizing one half of a binding pair (e.g., streptavidin) to the support and the other half (e.g., biotin) to the protein. In some embodiments, the solid support comprises glass and the protein or protein fragment is immobilized via physical adsorption.

The conditions sufficient to form antibody/CNR complexes and antibody/SARS-CoV-2 protein complexes are not particularly restricted but can be the same as those in routine use for conventional immunoassays. A typical procedure can comprise, for example, incubating or allowing to stand said sample and said SARS-CoV-2 protein or protein fragment and/or said CNR together at a temperature of generally not higher than about 45° C. In some embodiments, the temperature is about 4° C. to about 40° C. In some embodiments, the temperature is about 20° C. to about 40° C. or about 25° C. to about 40° C. (e.g., about 25° C., 30° C., 35° C. or about 40° C.). In some embodiments, the sample and the SARS2-CoV-2 protein or protein fragment and/or the CNR are incubated for about 0.5 hours to about 40 hours. In some embodiments, the incubation time is about 1 hours to about 20 hours (e.g., about 1, 2, 3, 4, 5, 6, 7, 8, 9, 10, 11, 12, 13, 14, 15, 16, 17, 18, 19, or about 20 hours). In some embodiments, the sample can be diluted using a conventional buffer, e.g., in the pH range of about 5 to about 9, such as, but not limited to citrate buffer, phosphate buffer, tris buffer, acetate buffer, etc. In some embodiments, a blocking agent (e.g., bovine serum albumin) can be used to further reduce non-specific binding of sample antibodies to the target antigen.

As described hereinabove, the sample can be incubated with the CNR prior to, at the same time as, or after the sample is incubated with the SARS-CoV-2 protein or protein fragment. In some embodiments, the CNR is incubated with the sample at the same time as the SARS-CoV-2 protein or protein fragment. Thus, for example, in some embodiments, the CNR can be added to the sample and the mixture incubated together with the SARS-CoV-2 protein or protein fragment. Any antibody/CNR complexes that form can be removed to provide a subtracted sample and a detection reagent can be added to detect any antibody/SARS-CoV-2 complexes.

In some embodiments, the CNR is incubated with the sample prior to the incubation of the sample with the SARS-CoV-2 protein or protein fragment under conditions suitable for antibody/CNR complexes to form if a suitable antibody is present in the sample, and any antibody/CNR complexes that are formed (i.e., between the CNR and sample antibodies that bind to common coronavirus epitopes or epitopes unique to common coronaviruses) can be removed (e.g., using a tag associated with the CNR) to provide the subtracted sample. The subtracted sample can then be incubated with the SARS-CoV-2 antigen. Alternatively, the unsubtracted sample that includes the CNR and any antibody/CNR complexes that have formed can be incubated with the SARS-CoV-2 antigen, and the CNR and any antibody/CNR complexes can be removed (e.g., via washing) after incubation of the unsubtracted sample with the SARS-CoV-2 antigen and prior to detection of the antibody/SARS-CoV-2 antibodies.

In some embodiments, such as shown in FIG. 1C, the sample is incubated with the SARS-CoV-2 protein or protein fragment and then any antibodies not complexed to the SARS-CoV-2 protein or protein fragment are removed using a washing step. For example, when the SARS-CoV-2 protein or protein fragment is immobilized on a solid support, the sample can be contacted to the solid support and incubated with the SARS-CoV-2 protein or protein fragment under conditions sufficient for antibody/SARS-CoV-2 protein complexes to form between antibodies in the sample that bind to common or unique epitopes in the SARS-CoV-2 protein or protein fragment. Then, the solid support can be washed (e.g., removing any antibodies that do not bind common coronavirus epitopes or unique SARS-CoV-2 epitopes). The solid support (now containing sample antibodies bound to the immobilized SARS-CoV-2 protien) can be contacted with a solution comprising the CNR and the solution comprising the CNR can be incubated with the solid support under conditions sufficient for antibody/CNR complexes to form between the CNR and any sample antibodies that bind to common coronavirus epitopes.

In some embodiments, either or both of the incubation steps can be performed at a temperature between about 4° C. and about 45° C. for about 0.5 hours to about 40 hours. In some embodiments, the temperature is about 20° C. to about 40° C. (e.g., about 20° C., 25° C., 30° C., 35° C. or about 40° C.) or about 25° C. to about 40° C. In some embodiments, incubation is performed for about 1 hour to about 20 hours (e.g., about 1, 2, 3, 4, 5, 6, 7, 8, 9, 10, 11, 12, 13, 14, 15, 16, 17, 18, 19 or about 20 hours).

The amount of SARS-CoV-2 protein or protein fragment is not particularly restricted but can generally be a typical amount of antigen used in immunoassays. In some embodiments, the amount of SARS-CoV-2 protein or protein fragment used is in excess of the amount of antibody present or suspected of being present in the sample. In some embodiments, the amount of SARS-CoV-2 protein or protein fragment can be about 0.01 μg to about 10 μg or about 0.1 μg to about 1 μg (e.g., for a 100 μl sample volume). In some embodiments, the amount of CNR (or the amount of each CNR if a mixture is used) can be about 0.1 μg to about 100 μg. In some embodiments, the amount of CNR is about 0.5 μg to about 50 μg.

The solid support can be washed again to remove any antibody/CNR complexes that have formed (i.e., forming the subtracted sample). After the unbound substances (including any antibody/CNR complexes and uncomplexed CNR) not coupled to the solid phase antigen are removed, an antibody assay or detection reagent can be added to detect any antibody/SARS-CoV-2 protein complexes. These complexes can be detected or quantitated by a detection means corresponding to the particular assay or detection reagent. Thus, in some embodiments, the analyzing comprises contacting the subtracted sample with a detection reagent and detecting a signal associated with the detection reagent and/or any product formed between the detection reagent and the antibody/SARS-CoV-2 protein complex.

Any suitable detection reagent for use in immunoassays can be used. Suitable reagents can be prepared via methods known in the art and/or can be purchased from commercial sources. In some embodiments, the detection reagent is a compound that can form a directly detectable product with the target antibody. In some embodiments, the detection reagent can form a product that can be indirectly detected, by means of adding a supplementary reagent, such as an enzyme substrate.

In some embodiments, the detection reagent is a secondary antibody. The secondary antibody can be an antibody that binds to the Fc region of the target antibody. For example, in the case of human samples, the secondary antibody can be an antibody that specifically binds to the Fc region of human IgG, IgA, or IgM antibodies. Such anti-human immunoglobulin antibodies include anti-sera and the purified products there (i.e., polyclonal antibodies), as well as monoclonal antibodies. The antibodies are available from various animals (e.g., mice, rats, rabbits, goats, etc.) immunized using an immunoglobulin in the class corresponding to the target antibody as an immunogen. In some embodiments, the secondary antibody is an Fc-specific anti-IgG antibody. The secondary antibody can also include a detectable label, such as a radioisotope (e.g., ¹²⁵I, ³H, ¹⁴C, etc.), an enzyme, such as alkaline phosphatase (ALP) or a peroxidase (e.g., horse radish peroxidase (HRP); a fluorescent substance such as fluorescein isothiocyanate (FITC), tetramethylrhodamine isothiocyanate (RITC), etc.; or 1N-(2,2,6,6-tetramethyl-1-oxyl-4-piperidyl)-5N-(aspartate)-2,4-dinitrobenzene (TOPA), etc. The immunoassay methods using the above-mentioned labels are called radioimmunoassay, enzyme immunoassay, fluoroimmunoassay, and spin immunoassay, respectively. An immunochromatoassay method using an antibody assay reagent prepared by labeling colloidal gold-stained latex particles can also be employed.

In some embodiments, the secondary antibody detection reagent is incubated or allowed to stand with any antibody-antigen complexes in the subtracted sample using the same conditions as described hereabove with regard to contact of the sample and the SARS-CoV-2 protein or protein fragment, i.e., a temperature that is about 45° C. or less (e.g., about 4° C. to about 40° C.) for about 0.5 hours to about 40 hours (e.g., about 1 hour to about 20 hours).

The presence or absence of the target antibody (present in an antibody/SARS-CoV-2 protein complex) is then evaluated by measuring the label activity, which depends on the kind of label used in the detection reagent (or the indirect label that is used with the detection reagent), in the routine manner or in terms of the antibody titer calculated from the measured value. For example, depending upon the type of label, measurement can be performed using a colorimeter, a fluorophotometer, or a photon counter. In some embodiments, the signal provided by the measurement can be compared to a signal provided by a control sample with a known concentration of the detectable label or a standard curve prepared by measuring signal from a plurality of samples comprising a plurality of known concentrations of the detectable label. In some embodiments, for example, the detection regent is a secondary antibody labeled with HRP. The amount of target antibody can be detected indirectly by contacting the HRP (attached to the secondary antibody complexed to the target antibody/immobilized SARS-CoV-2 protein or protein fragment complex) with a substrate that forms a chromogenic, fluorescent, or luminescent product and measuring the amount of product. In some embodiments, the detecting comprises measuring a signal from an assay performed on a plurality of samples where the plurality of samples comprise a series of sample prepared by diluting an original sample (e.g., using a suitable buffer in which antibodies are stable).

### III.B. Common Epitope Disrupted Mutants (CEDM)

In some embodiments, e.g., in case CNR incubation sacrifices an undesirable level of sensitivity, the presently disclosed subject matter can relate to the use of Common Epitope Disrupted Mutants (CEDM). Coronavirus proteins (e.g, coronavirus N and S proteins) have several conserved regions that are consistent across coronavirus strains. As such, recombinant mutant versions of the SARS-CoV-2 proteins (e.g., the N or S proteins) can be engineered to lack the commonly conserved domains. These mutants can maintain epitopes specific to SARS-CoV-2 but lack common cross-reactive epitopes. Instead of the wild type proteins, the CEDM can serve as the target antigen for anti-SARS-CoV-2 antibodies in a sample, thus increasing specificity. Like the CNR strategy, the CEDM strategy runs can decrease sensitivity due to removal of some target epitopes. However, CEDMs can be combined (e.g., using mixtures of mutant N and/or S proteins) as target antigens in an immunoassay, increasing signal to noise ratio, increasing sensitivity while maintaining specificity gained from the recombinant mutant proteins. Such CEDM antigens can be employed in any assay in either native form or denatured form (e.g. ELISA or Western Blot), depending upon the question(s) being asked.

In some embodiments, the CEDM is a mutant of a SARS-CoV-2 nucleocapsid protein. Coronavirus nucleocapsid proteins have a number of conserved regions and renders the native SARS-CoV-2 N proteins subject to cross-reactivity with antibodies to common cold coronaviruses, e.g., as described hereinabove. Common nucleocapsid protein epitopes that are found in the common coronaviruses include: GQGVPI (SEQ ID NO: 31); RNLVPI (SEQ ID NO. 32); PRWYFYYLGTGP (SEQ ID No. 33); PKVHFYYLGTGP (SEQ ID NO. 34); PKLHFYYLGTGP (SEQ ID NO. 35); KPRQKR (SEQ ID NO. 36); and KPRWKR (SEQ ID NO. 37). In some embodiments, the CEDM is a SARS-CoV-2 nucleocapsid protein (e.g., of SEQ ID NO: 12 or a fragment thereof) wherein one or more of these common epitopes has been deleted.

For example, the SARS-CoV-2 N protein comprising the sequence of SEQ ID NO: 12 includes each of the following three common epitopes: GQGVPI (SEQ ID NO: 31); PRWYFYYLGTGP (SEQ ID NO: 33); and KPRQKR (SEQ ID NO: 36). These three common epitopes are underlined in sequence for SEQ ID NO: 12 here:

MSDNGPQNQRNAPRITFGGPSDSTGSNQNGERSGARSKQRRPQGLPN
NTASWFTALTQHGKEDLKFPRGQGVPINTNSSPDDQIGYYRRATRRI
RGGDGKMKDLSPRWYFYYLGTGPEAGLPYGANKDGIIWVATEGALNT
PKDHIGTRNPANNAAIVLQLPQGTTLPKGFYAEGSRGGSQASSRSSS
RSRNSSRNSTPGSSRGTSPARMAGNGGDAALALLLLDRLNQLESKMS
GKGQQQQGQTVTKKSAAEASKKPRQKRTATKAYNVTQAFGRRGPEQT
QGNFGDQELIRQGTDYKHWPQIAQFAPSASAFFGMSRIGMEVTPSGT
WLTYTGAIKLDDKDPNFKDQVILLNKHIDAYKTFPPTEPKKDKKKKA
DETQALPQRQKKQQTVTLLPAADLDDFSKQLQQSMSSADSTQA

Thus, in some embodiments, the resulting mutant SARS-CoV-2 N protein (i.e., the CEDM) comprises, consists essentially of, or consists of the amino acid sequence of SEQ ID NO: 12 wherein the amino acid sequence of one, two or three of SEQ ID NOS 31, 33, and 36 are deleted, or a fragment thereof (i.e., a fragment of the common epitope deleted sequence). In some embodiments, all three common epitopes are deleted and the CEDM comprises, consists essentially of, or consists of the following sequence:

(SEQ ID NO: 51)
MSDNGPQNQRNAPRITFGGPSDSTGSNQNGERSGARSKQRRPQGLPN
NTASWFTALTQHGKEDLKFPRNTNSSPDDQIGYYRRATRRIRGGDGK
MKDLSEAGLPYGANKDGIIWVATEGALNTPKDHIGTRNPANNAAIVL
QLPQGTTLPKGFYAEGSRGGSQASSRSSSRSRNSSRNSTPGSSRGTS
PARMAGNGGDAALALLLLDRLNQLESKMSGKGQQQQGQTVTKKSAAE
ASKTATKAYNVTQAFGRRGPEQTQGNFGDQELIRQGTDYKHWPQIAQ
FAPSASAFFGMSRIGMEVTPSGTWLTYTGAIKLDDKDPNFKDQVILL
NKHIDAYKTFPPTEPKKDKKKKADETQALPQRQKKQQTVTLLPAADL
DDFSKQLQQSMSSADSTQA

In some embodiments, the CEDM comprises, consists essentially of, or consists of a fragment of SEQ ID NO: 51. In some embodiments, the CEDM comprises, consists essentially of, or consists of an amino acid sequence having about 80%, about 85%, about 90%, about 95%, about 96%, about 97%, about 98%, or about 99% homology to SEQ ID NO: 51 or a fragment thereof.

Alternatively, in some embodiments, the CEDM is a mutant of a SARS-CoV-2 spike protein. Common epitopes among the common coronavirus spike proteins include the following: RSAIEDLLF (SEQ ID NO: 38); RSFIEDLLF (SEQ ID NO: 39); RSFFEDLLF (SEQ ID NO: 40); VLPPLL (SEQ ID NO: 41); VLPPIL (SEQ ID NO: 42); NQKLIA (SEQ ID NO: 43); ILSRLD (SEQ ID NO: 44); QIDRLI (SEQ ID NO: 45); KWPWY (SEQ ID NO: 46); KVNECVKSQS (SEQ ID NO: 47); FCGNG (SEQ ID NO: 48); KWPWYVWL (SEQ ID NO: 49); and KWPWWVWL (SEQ ID NO: 50). In some embodiments, the CEDM comprises a SARS-CoV-2 spike protein wherein one or more of SEQ ID NOS: 38-50 have been deleted. In some embodiments, the CEDM is free of any of SEQ ID NOS: 38-50. In some embodiments, the CEDM is a SARS-CoV-2 spike protein having a sequence comprising, consisting essentially of, or consisting of one of SEQ ID NOS: 22-30, or a fragment thereof, wherein the amino acid sequence of one or more of SEQ ID NOS: 38-50 have been deleted.

For example, common spike protein epitopes of SEQ ID NOS: 39, 41, 43, 44, 45, and 46 are underlined in SEQ ID NO: 22 (SARS-CoV-2 spike protein, Wuhan isolate):

MFVFLVLLPLVSSQCVNLTTRTQLPPAYTNSFTRGVYYPDKVFRSSV
LHSTQDLFLPFFSNVTWFHAIHVSGTNGTKRFDNPVLPFNDGVYFAS
TEKSNIIRGWIFGTTLDSKTQSLLIVNNATNVVIKVCEFQFCNDPFL
GVYYHKNNKSWMESEFRVYSSANNCTFEYVSQPFLMDLEGKQGNFKN
LREFVFKNIDGYFKIYSKHTPINLVRDLPQGFSALEPLVDLPIGINI
TRFQTLLALHRSYLTPGDSSSGWTAGAAAYYVGYLQPRTFLLKYNEN
GTITDAVDCALDPLSETKCTLKSFTVEKGIYQTSNFRVQPTESIVRF
PNITNLCPFGEVFNATRFASVYAWNRKRISNCVADYSVLYNSASFST
FKCYGVSPTKLNDLCFTNVYADSFVIRGDEVRQIAPGQTGKIADYNY
KLPDDFTGCVIAWNSNNLDSKVGGNYNYLYRLFRKSNLKPFERDIST
EIYQAGSTPCNGVEGFNCYFPLQSYGFQPTNGVGYQPYRVVVLSFEL
LHAPATVCGPKKSTNLVKNKCVNFNFNGLTGTGVLTESNKKFLPFQQ
FGRDIADTTDAVRDPQTLEILDITPCSFGGVSVITPGTNTSNQVAVL
YQDVNCTEVPVAIHADQLTPTWRVYSTGSNVFQTRAGCLIGAEHVNN
SYECDIPIGAGICASYQTQTNSPRRARSVASQSIIAYTMSLGAENSV
AYSNNSIAIPTNFTISVTTEILPVSMTKTSVDCTMYICGDSTECSNL
LLQYGSFCTQLNRALTGIAVEQDKNTQEVFAQVKQIYKTPPIKDFGG
FNFSQILPDPSKPSKRSFIEDLLFNKVTLADAGFIKQYGDCLGDIAA
RDLICAQKFNGLTVLPPLLTDEMIAQYTSALLAGTITSGWTFGAGAA
LQIPFAMQMAYRFNGIGVTQNVLYENQKLIANQFNSAIGKIQDSLSS
TASALGKLQDVVNQNAQALNTLVKQLSSNFGAISSVLNDILSRLDKV
EAEVQIDRLITGRLQSLQTYVTQQLIRAAEIRASANLAATKMSECVL
GQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDV
VIGIVNNTVYDPLQPELDSFKEELDKYFKNHTSPDVDLGDISGINAS
VVNIQKEIDRLNEVAKNLNESLIDLQELGKYEQYIKWPWYIWLGFIA
GLIAIVMVTIMLCCMTSCCSCLKGCCSCGSCCKFDEDDSEPVLKGVK
LHYT

Thus, in some embodiments, the resulting mutant SARS-CoV-2 N protein (i.e., the CEDM) comprises, consists essentially of, or consists of the amino acid sequence of SEQ ID NO: 22 wherein amino acid sequences of one, two, three, four, five or all six of SEQ ID NOS 39, 41, 43, 44, 45, and 46 are deleted, or a fragment thereof (i.e., a fragment of the CEDM sequence). In some embodiments, the CEDM comprises, consists essentially of, or consists of an amino acid sequence having at least about 70% (e.g., at least about 70%, about 75%, about 80%, about 85%, about 90% or at least about 95% homology) to the amino acid of SEQ ID NO: 22 wherein amino acid sequences of one, two, three, four, five or six of SEQ ID NOS: 39, 41, 43, 44, 45, and 46 have been deleted, or a fragment thereof.

The deletions described herein are based upon linear amino acid sequence; such that changes could alter tertiary structure. As such, the mutant protein can be empirically tested in serologic assays that make use of native conformation (e.g. ELISA) using a sample known to contain authentic anti-SARS-CoV-2 antibodies to verify that the CEDM maintains a suitable structure to bind to anti-SARS-CoV-2 antibodies in such assays. In some embodiments, corresponding wild-type protein can be used as a comparative positive control. Anti-SARS-CoV-2 antibodies can be purchased from commercial sources. For example, an anti-SARS-CoV-2 anti-nucleocapsid polyclonal antibody is available from Native Antigen Company (Kidlington, United Kingdom, catalog number PAB21474. Anti-SARS-CoV-2 anti-spike (51) and anti-spike (S2) protein polyclonal antibodies are also available from Native Antigen Company (Kidlington, United Kingdom, catalog numbers PAB21471 and PAB21472, respectively). Monoclonal antibodies to these proteins can also be purchased, for example, from Sigma-Aldrich (St. Louis, Mo., United States of America). Western Blot analysis uses denatured epitopes and would not be expected to be significantly affected by using the mutant protein.

Accordingly, in some embodiments, the presently disclosed subject matter provides a method of performing an immunoassay to detect a presence or absence of an antibody for severe acute respiratory syndrome coronavirus 2 (SARS-CoV-2) in a sample comprising one or more antibodies, the method comprising: incubating the sample with a mutant protein or protein fragment for SARS-CoV-2 under conditions sufficient to form an antibody/mutant protein complex between the mutant protein or protein fragment for SARS-CoV-2 and an antibody in the sample specific for said mutant protein or protein fragment for SARS-CoV-2, wherein said mutant protein or protein fragment for SARS-CoV-2 comprises or consists of an amino acid sequence of a SARS-CoV-2 protein (e.g., a N or S protein) wherein one or more common epitope sequences has been deleted (i.e., is a CEDM); and analyzing the sample to determine the presence or absence of an antibody/mutant protein complex (i.e., antibody/CEDM complex), thereby determining the presence or absence of an antibody in the sample specific for SARS-CoV-2.

In some embodiments, the mutant protein or protein fragment for SARS-CoV-2 is a common epitope deleted mutant nucleocapsid protein or a fragment thereof, wherein said common epitope deleted mutant nucleocapsid protein is a recombinant protein having an amino acid sequence of a nucleocapsid protein of SARS-CoV-2 wherein one or more common coronavirus nucleocapsid protein epitope has been removed. In some embodiments, each of said one or more common coronavirus nucleocapsid protein epitope has an amino acid sequence selected from SEQ ID Nos 31-37. In some embodiments, the mutant protein or protein fragment for SARS-CoV-2 is a common epitope deleted mutant spike protein or a fragment thereof, wherein said common epitope deleted mutant spike protein is a recombinant protein having an amino acid sequence of a spike protein of SARS-CoV-2 wherein one or more common coronavirus spike protein epitope has been removed, wherein each of said one or more common coronavirus spike protein epitope has an amino acid sequence selected from the group comprising SEQ ID Nos: 38-50. In some embodiments, the mutant protein or protein fragment for SARS-CoV-2 comprises a combination or mixture of a common epitope deleted mutant spike protein or protein fragment and a common epitope deleted mutant nucleocapsid protein or protein fragment.

The sample can be any sample described above with regard to methods involving CNRs. In some embodiments, the sample can be a blood sample or a serum sample (e.g., from a patient suspected of having had COVID-19 or having been exposed to SARS-CoV-2). In some embodiments, the mutant protein or protein fragment for SARS-CoV-2 (i.e., the CEDM) is immobilized on a solid support (e.g., a microtiter plate).

The conditions sufficient for antibody/mutant protein complexes to form can generally be the same as the incubation conditions described hereinabove with regard to methods involving CNRs and/or that are in routine use for conventional immunoassays. For example, in some embodiments, the incubating can be performed at a temperature not higher than about 45° C. In some embodiments, the temperature is about 4° C. to about 40° C. In some embodiments, the temperature is about 20° C. to about 40° C. or about 25° C. to about 40° C. (e.g., about 25° C., about 30° C., about 35° C. or about 40° C.). In some embodiments, the sample and the CEDM are incubated together for about 0.5 hours to about 40 hours. In some embodiments, the incubation time is about 1 hours to about 20 hours (e.g., about 1, 2, 3, 4, 5, 6, 7, 8, 9, 10, 11, 12, 13, 14, 15, 16, 17, 18, 19, or about 20 hours). In some embodiments, the sample can be diluted using a conventional buffer, e.g., in the pH range of about 5 to about 9, such as, but not limited to citrate buffer, phosphate buffer, tris buffer, acetate buffer, etc. In some embodiments, a blocking agent (e.g., bovine serum albumin) can be used to further reduce non-specific binding of sample antibodies to the target antigen. The amount of CEDM can generally be the same as the amount of antigen used for methods involving CNRs.

The analyzing can be performed as described hereinabove with regard to methods involving CNRs. Thus, the analyzing can be performed by contacting the incubated sample (e.g., after a washing step to remove unbound antibodies from the sample when the CEDM is immobilized on a solid support) with a suitable detection reagent and detecting a signal associated with the reagent or a product thereof. In some embodiments, the detection reagent is a secondary antibody that binds the Fc region of the antibodies in the sample (e.g., the Fc region of human antibodies) wherein the secondary antibody is further labeled with a detectable label (e.g., a radioisotope, an enzyme that catalyzes the reaction of a chromogenic substrate, a fluorophore, etc.). Suitable secondary antibodies are commercially available from a variety of sources.

In some embodiments, the mutant protein or protein fragment for SARS-CoV-2 is a mutant spike protein, wherein the mutant spike protein has an amino acid sequence selected from SEQ ID NOs: 22-30 or a fragment thereof from which one or more common coronavirus spike protein epitope (e.g., SEQ ID NOS: 38-50) have been removed. In some embodiments, any common epitopes present in the SARS-CoV-2 wild-type protein are deleted.

In some embodiments, the mutant protein or protein fragment for SARS-CoV-2 is a mutant nucleocapsid protein, wherein the mutant nucleocapsid protein has an amino acid sequence of SEQ ID NO: 12 from which one or more common coronavirus nucleocapsid protein epitope has been removed. In some embodiments, the mutant protein or protein fragment has a sequence of SEQ ID NO: 12 from which one, two, or three of SEQ ID NOS: 31, 33, and 36 have been deleted, or a fragment thereof. In some embodiments, the mutant protein has an amino acid sequence that has at least about 95% homology to the amino acid sequence of SEQ ID NO: 12 wherein one, two or three of the sequences of SEQ ID NOS; 31, 33, and 36 have been removed. In some embodiments, the mutant protein has an amino acid sequence of SEQ ID NO: 51 or a fragment thereof or an amino acid sequence having at least about 95% homology to said sequence or a fragment thereof.

In some embodiments, the presently disclosed subject matter provides a CEDM of a SARS-CoV-2 protein or protein fragment. In some embodiments, the CEDM is a mutant of a N or S SARS-CoV-2 protein or protein fragment. In some embodiments, the CEDM has an amino acid sequence comprising, consisting essentially of, or consisting of one of SEQ ID NO: 22-30 or a fragment thereof from which one or more (or all) amino acid sequences of SEQ ID NOS. 38-50 have been deleted; or an amino acid sequence having at least about 70%, about 75%, about 80%, about 85%, about 90%, or about 95% homology to said sequence. In some embodiments, the CEDM has an amino acid sequence comprising, consisting essentially of, or consisting of a SARS-CoV-2 nucleocapsid protein or protein fragment from which one or more (or all) amino acid sequences of SEQ ID NOS. 31-37 have been deleted, or an amino acid sequence having at least about 70%, about 75%, about 80%, about 85%, about 90%, or about 95% homology to said sequence. In some embodiments, the CEDM has an amino acid sequence comprising, consisting essentially of, or consisting of SEQ ID NO: 12 from which amino acid sequences of one or more of SEQ ID NO: 31, 33, and 36 have been deleted, or a fragment thereof, or an amino acid sequence having at least about 70%, about 75%, about 80%, about 85%, about 90%, or about 95% homology thereto. In some embodiments, the CEDM has an amino acid sequence comprising, consisting essentially of, or consisting of SEQ ID NO: 51, or a fragment thereof, or an amino acid sequence having at least about 70%, about 75%, about 80%, about 85%, about 90%, or about 95% to said sequence.

The CEDMs can be prepared via recombinant methods known in the art. In some embodiments, the CEDM (i.e., the mutant protein) can further comprise a tag, such as, but not limited to a GST tag, a His tag, a FLAG tag, a HA tag, a cMyc tag, an ALFA tag, a V5 tag, a Spot tag, a T7 tag, an NE tag and any combination thereof. The tag(s) can be attached at the N-terminal or C-terminal ends of the CEDM, optionally via an amino acid sequence that can be cleaved by a protease. In some embodiments, the CEDM can be immobilized on a solid support, such as one of the solid supports described hereinabove (e.g., a microtiter plate).

In some embodiments, the presently disclosed subject matter provides a kit for performing an assay for detecting the presence or absence of a SARS-CoV-2 antibody in a sample wherein the kit comprises one or more of the presently disclosed CEDM. In some embodiments, the CEDM comprises, consists essentially of, or consists of SEQ ID NO: 51, or a fragment thereof, or an amino acid sequence having at least about 70%, about 75%, about 80%, about 85%, about 90%, or about 95% homology to SEQ ID NO: 51 or a fragment thereof. In some embodiments, the kit is for performing an immunoassay and the mutant protein is immobilized on a solid support. For example, in some embodiments, the kit comprises a microtiter plate having the CEDM immobilized in one or more wells of the microtiter plate. In some embodiments, the kit can include written instructions for performing the assay.

In some embodiments, the kit can further comprise one or more additional reagent, control species or buffers for performing the assay. For instance, in some embodiments, the kit can further comprise a detection reagent. In some embodiments, the detection reagent comprises a labeled reporter antibody (i.e., a secondary antibody) that binds to a constant region of an antibody. In some embodiments, the labeled reporter antibody binds to the constant region of human IgG antibodies. In some embodiments, e.g., if the detection reagent comprises an enzyme, the one or more additional reagents can include an enzyme substrate for the enzyme. In some embodiments, the kit can include one or more wild-type SARS-CoV-2 protein for use as a control. In some embodiments, the kit can include one or more blocking agent (e.g., bovine serum albumin).

In some embodiments, the presently disclosed subject matter provides an immunoassay device (e.g., a lateral flow device or a microtiter plate) comprising a CEDM as described herein (e.g., the mutant protein comprising SEQ ID NO: 51).

### III.C. Use of Common Coronavirus Proteins to Establish Background

In some embodiments, the presently disclosed subject matter provides a method that takes a methodological approach in which each sample (e.g., each serum specimen) is evaluated against antigens from multiple coronaviruses, e.g., SARS-CoV-2 and at least one common coronavirus (e.g., OC43-CoV, HKU1-CoV, NL63-CoV, and/or 229E-CoV). Results can be compared between the results from evaluation with the SARS-CoV-2 antigen and the common coronavirus antigen(s) to determine establish a background signal for sample reactivity with coronaviruses.

For example, the method can comprise splitting a sample (e.g., a serum or blood sample) into 5 aliquots. A multi-well plate with 5 distinct wells can be prepared where one well contains a SARS-CoV-2 antigen (e.g., a SARS-CoV-2 N or S protein), while the other 4 wells containing the analogous protein from OC43, HKU1, NL63 and 229E. A true positive for SARS-CoV-2 would demonstrate higher signal in the SARS-CoV-2 well than in any of the other wells. This approach can involve a careful understanding of the linear dynamic range of the assay, and possible dilution (titration) of samples that give signal above the range. On the other hand, this maneuver can increase the specificity of SARS-CoV-2 serology.

Accordingly, in some embodiments, the presently disclosed subject matter provides a method of performing an immunoassay to detect a presence or absence of an antibody for SARS-CoV-2 (i.e., a “COVID-19 antibody”) in a sample comprising one or more antibodies, wherein the method comprises: receiving a sample comprising antibodies (e.g., from a patient suspected of having been exposed to SARS-CoV-2); splitting the sample into a plurality of aliquots (e.g., two to five aliquots); incubating one of the plurality of aliquots (e.g., one of the two to five aliquots) with a viral protein from SARS-CoV-2 or a fragment thereof under conditions sufficient to form antibody/protein complexes between the viral protein or fragment thereof and any antibody in the sample specific for the viral protein; incubating each remaining aliquot of the plurality of aliquots (e.g., each of the remaining aliquots of the two to five aliquots) with a corresponding viral protein or fragment thereof from a different common coronavirus under conditions sufficient to form antibody/protein complexes between the corresponding viral protein or fragment thereof and any antibody in the sample specific for the corresponding viral protein; determining a signal associated with antibody binding for each of the plurality of aliquots (e.g., each of the two to five aliquots), thereby determining a plurality of binding signals for the sample, wherein each of the plurality of binding signals is for a different viral protein; and comparing the binding signals. In some embodiments, the common coronavirus is selected from OC43-CoV, HKU1-CoV, NL63-CoV, and 229E-CoV.

In some embodiments, the viral protein from SARS-CoV-2 and each corresponding viral protein is a spike protein or fragment thereof or a nucleocapsid protein or fragment thereof. For example, in some embodiments, the viral protein from SARS-CoV-2 can have an amino acid sequence of one of SEQ ID NOS. 12 and 22-30, or a fragment thereof. In some embodiments, the corresponding protein can have an amino acid sequence of one of SEQ ID NOS. 2, 4, 6, 8, 10, 14, 16, 18, and 20, or a fragment thereof.

The sample can be any sample as described above regarding the methods involving CNRs or CEDMs. In some embodiments, the sample is a blood sample or a serum sample. Incubation and detection methods can be the same as those described above with regard to the CNR and CEDM methods.

In some embodiments, the sample is split into two, three, four or five aliqouts. In some embodiments, the sample is split into five aliquots and a binding signal is determined for SARS-CoV-2 and each of OC43-CoV, HKU1-CoV, NL63-CoV, and 229E-CoV.

### REFERENCES

All references listed below, as well as all references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.
Altschul et al. Basic local alignment search tool. J. Mol. Biol. 1990a; 215:403-410.
Altschul et al. Protein database searches for multiple alignments. Proc Natl Acad Sci USA 1990b; 87:14:5509-5513.
Altschul et al. Gapped BLAST and PSI-BLAST: a new generation of protein database search programs. Nucleic Acids Res. 1997; 25:3389-3402.
Ausubel et al. Current Protocols in Molecular Biology, Greene Publishing, 1995.
Devereux et al. A comprehensive set of sequence analysis programs for the VAX. Nuc. Acids Res. 1984; 12:387-395.
Gait. Oligonucleotide Synthesis: A Practical Approach, IRL Press, Oxford, England, 1984.
Glover. DNA Cloning: A Practical Approach. Oxford Press, Oxford, England, 1985.
Gorse G J, Patel G B, Vitale J N, O'Connor T Z. Prevalence of antibodies to four human coronaviruses is lower in nasal secretions than in serum. Clin Vaccine Immunol. 2010; 17(12): 1875-1880.
Harlow & Lane, Antibodies, A Laboratory Manual, Cold Spring Harbor, N.Y., United States of America, 1988.
Karlin & Altschul. Methods for assessing the statistical significance of molecular sequence features by using general scoring schemes. Proc Natl Acad Sci USA 1990; 87:2264-2268.
Karlin & Altschul. Applications and statistics for multiple high-scoring segments in molecular sequences. Proc Natl Acad Sci USA 1993; 90:5873-5877.
Meyer B, Drosten C, Muller M A. Serological assays for emerging coronaviruses: challenges and pitfalls. Virus Res. 2014; 194:175-183.
Patrick D M, Petric M, Skowronski D M, et al. An Outbreak of Human Coronavirus OC43 Infection and Serological Cross-reactivity with SARS Coronavirus. Can J Infect Dis Med Microbiol. 2006; 17(6):330-336.
Roe et al. DNA Isolation and Sequencing: Essential Techniques, John Wiley, New York, N.Y., United States of America, 1996.
Sambrook et al. Molecular Cloning: A Laboratory Manual, Cold Spring Harbor Laboratory Publications, Cold Spring Harbor, N.Y., United States of America, 1989.
Su S, Wong G, Shi W, et al. Epidemiology, Genetic Recombination, and Pathogenesis of Coronaviruses. Trends Microbiol. 2016; 24(6):490-502.

It will be understood that various details of the presently disclosed subject matter may be changed without departing from the scope of the presently disclosed subject matter. Furthermore, the foregoing description is for the purpose of illustration only, and not for the purpose of limitation.

1. A method of performing an immunoassay to detect a presence or absence of an antibody for severe acute respiratory syndrome coronavirus 2 (SARS-CoV-2) in a sample comprising one or more antibodies, the method comprising:
incubating the sample with a cross-reactivity neutralizing reagent (CNR) comprising, consisting essentially of, or consisting of one or more proteins or protein fragments from a common coronavirus, wherein said one or more proteins or protein fragments are selected from the group consisting of a spike protein, a spike protein fragment, a nucleocapsid protein, and a nucleocapsid protein fragment, wherein the incubating is performed under conditions sufficient to form an antibody/CNR complex between the CNR and any antibody present in the sample specific for the CNR;
incubating the sample with a SARS-CoV-2 protein or a fragment thereof under conditions sufficient to form an antibody/SARS-CoV-2 protein complex between the SARS-CoV-2 protein or fragment thereof and any antibody in the sample specific for the SARS-CoV-2 protein or fragment thereof;
treating the sample to remove any antibody/CNR complex present in the sample, thereby forming a subtracted sample; and
analyzing the subtracted sample to determine the presence or absence of any antibody/SARS-CoV-2 protein complex, thereby detecting a presence or absence of antibody binding to the SARS-CoV-2 protein or fragment thereof.

2. The method of claim 1, wherein the sample is a blood sample or a serum sample.

3. The method of claim 1, wherein the CNR comprises, consists essentially of, or consists of one or more recombinant proteins or protein fragments.

4. The method of claim 1, wherein the common coronavirus is selected from the group consisting of coronavirus OC43 (OC43-CoV), coronavirus HKU1 (HKU1-CoV), coronavirus NL63 (NL63-CoV), and coronavirus 229E (229E-CoV).

5. The method of claim 1, wherein the CNR comprises, consists essentially of, or consists of one or more nucleocapsid proteins or protein fragments.

6. The method of claim 1, wherein the CNR comprises, consists essentially of, or consists of one of SEQ ID NOS. 2, 4, 6, 8, 10, 14, 16, 18, and 20.

7. The method of claim 1, wherein the SARS-CoV-2 protein or fragment thereof comprises or consists of one of SEQ ID NOS: 12 and 22-30.

8. The method of claim 1, wherein the SARS-CoV-2 protein of fragment thereof is immobilized on a solid support.

9. The method of claim 8, wherein the solid support is a microtiter plate.

10. A method of performing an immunoassay to detect a presence or absence of an antibody for severe acute respiratory syndrome coronavirus 2 (SARS-CoV-2) in a sample comprising one or more antibodies, the method comprising:
incubating the sample with a mutant protein or protein fragment for SARS-CoV-2 under conditions sufficient to form an antibody/mutant protein complex between the mutant protein or protein fragment for SARS-CoV-2 and an antibody in the sample specific for said mutant protein or protein fragment for SARS-CoV-2, wherein said mutant protein or protein fragment for SARS-CoV-2 comprises or consists of:
(i) a common epitope deleted mutant nucleocapsid protein or a fragment thereof, wherein said common epitope deleted mutant nucleocapsid protein is a recombinant protein having an amino acid sequence of a nucleocapsid protein of SARS-CoV-2 wherein one or more common coronavirus nucleocapsid protein epitope has been removed, wherein each of said one or more common coronavirus nucleocapsid protein epitope has an amino acid sequence selected from the group consisting of SEQ ID NOS: 31-37; or
(ii) a common epitope deleted mutant spike protein or a fragment thereof, wherein said common epitope deleted mutant spike protein is a recombinant protein having an amino acid sequence of a spike protein of SARS-CoV-2 wherein one or more common coronavirus spike protein epitope has been removed, wherein each of said one or more common coronavirus spike protein epitope has an amino acid sequence selected from the group consisting of SEQ ID NOS: 38-50; and
analyzing the sample to determine the presence or absence of an antibody/mutant protein complex, thereby determining the presence or absence of an antibody in the sample specific for SARS-CoV-2.

11. The method of claim 10, wherein the sample is a blood sample or a serum sample.

12. The method of claim 10, wherein the mutant protein or protein fragment for SARS-CoV-2 is a mutant spike protein, wherein the mutant spike protein has an amino acid sequence selected from the group consisting of SEQ ID NOS: 22-30 from which one or more common coronavirus spike protein epitope has been removed.

13. The method of claim 10, wherein the mutant protein or protein fragment for SARS-CoV-2 is a mutant nucleocapsid protein, wherein the mutant nucleocapsid protein has an amino acid sequence of SEQ ID NO: 12 from which one or more common coronavirus nucleocapsid protein epitope has been removed.

14. The method of claim 13, wherein the one or more common coronavirus nucleocapsid protein epitope is a peptide comprising an amino acid sequence selected from the group consisting of GQGVP (SEQ ID NO: 31), PRWYFYYLGTGP (SEQ ID NO: 33), and KPRQKR (SEQ ID NO: 36).

15. The method of one of claim 13, wherein the mutant protein comprises or consists of an amino acid having an amino acid sequence: MSDNGPQNQRNAPRITFGGPSDSTGSNQNGERSGARSKQRRPQGLPNNTASWFTA LTQHGKEDLKFPRNTNSSPDDQIGYYRRATRRIRGGDGKMKDLSEAGLPYGANKDGII WVVATEGALNTPKDHIGTRNPANNAAIVLQLPQGTTLPKGFYAEGSRGGSQASSRSSS RSRNSSRNSTPGSSRGTSPARMAGNGGDAALALLLLDRLNQLESKMSGKGQQQQG QTVTKKSAAEASKTATKAYNVTQAFGRRGPEQTQGNFGDQELIRQGTDYKHWPQIAQ FAPSASAFFGMSRIGMEVTPSGTWLTYTGAIKLDDKDPNFKDQVILLNKHIDAYKTFPP TEPKKDKKKKADETQALPQRQKKQQTVTLLPAADLDDFSKQLQQSMSSADSTQA (SEQ ID NO: 51), or a fragment thereof, or having an amino acid sequence having 95% homology to said amino acid sequence or a fragment thereof.

16. A mutant protein comprising, consisting essentially of, or consisting of the amino acid of SEQ ID NO: 51.

17. The mutant protein of claim 16, wherein the mutant protein further comprises a tag.

18. The mutant protein of claim 17, wherein the tag is selected from the group consisting of a glutathione-S-transferase (GST) tag, a His tag, a FLAG tag, a hemagglutinin (HA) tag, a cMyc tag, an ALFA-tag, a V5-tag, a Spot-tag, a T7-tag, an NE tag, and combinations thereof.

19. A kit for performing an immunoassay comprising the mutant protein of claim 16, wherein said mutant protein is immobilized on a solid support.

20. The kit of claim 19, wherein the kit further comprises a detection reagent, wherein the detection reagent comprises a labeled reporter antibody that binds to a constant region of an antibody.

21. A method of performing an immunoassay to detect a presence or absence of an antibody for severe acute respiratory syndrome coronavirus 2 (SARS-CoV-2) in a sample comprising one or more antibodies, the method comprising:
receiving a sample from a patient suspected of having been exposed to SARS-CoV-2;
splitting the sample into two to five aliquots;
incubating one of the two to five aliquots with a viral protein from SARS-CoV-2 or a fragment thereof under conditions sufficient to form antibody/protein complexes between the viral protein or fragment thereof and any antibody in the sample specific for the viral protein;
incubating each remaining aliquot of the two to five aliquots with a corresponding viral protein or fragment thereof from a different common coronavirus selected from the group consisting of coronavirus OC43 (OC43-CoV), coronavirus HKU1 (HKU1-CoV), coronavirus NL63 (NL63-CoV), and coronavirus 229E (229E-CoV) under conditions sufficient to form antibody/protein complexes between the corresponding viral protein or fragment thereof and any antibody in the sample specific for the corresponding viral protein;
determining a signal associated with antibody binding for each of the two to five aliquots, thereby determining a plurality of binding signals for the sample, wherein each of the plurality of binding signals is for a different viral protein; and
comparing the binding signals, thereby detecting the presence or absence of an antibody to SARS-CoV-2.

22. The method of claim 21, wherein the viral protein from SARS-CoV-2 and each corresponding viral protein is a spike protein or wherein each viral protein from SARS-CoV-2 and each corresponding viral protein is a nucleocapsid protein.

23. The method of claim 21, wherein splitting the sample into two to five aliquots comprises splitting the sample into five aliquots.
